(19) United States
(12) Patent Application Publication
(10) Pub. No.: US 2026/0315486 A1
(43) Pub. Date: Oct. 15, 2026

(54) ALECTINIB FOR THE TREATMENT OF ALK FUSION-POSITIVE SOLID OR CNS TUMOURS

(71) Applicant: Hoffmann-La Roche Inc., Little Falls, NJ (US)

(72) Inventors: Guillaume Jacques BERGTHOLD, Illkirch-GRAFFENSTADEN (FR); Hubertus Nicolaas CARON, Basel (CH); Clare Elizabeth DEVLIN, Hemel Hempstead (GB); Nassim DJEBLI, Rixheim (FR); Elena GUERINI, Basel (CH)

(73) Assignee: Hoffmann-La Roche Inc., Little Falls, NJ (US)

(21) Appl. No.: 19/677,440

(22) Filed: May 14, 2026

Related U.S. Application Data
(63) Continuation of application No. PCT/EP2024/082092, filed on Nov. 13, 2024.

(30) Foreign Application Priority Data
Nov. 15, 2023 (EP) 23210216.0

(51) Int. Cl. A61K 31/5377; A61P 35/00

(52) U.S. Cl. CPC A61K 31/5377; A61P 35/00

(57) ABSTRACT

The present invention relates to a method of treating an anaplastic lymphoma kinase (ALK) fusion-positive solid tumour or central nervous system tumour, comprising administering to a subject in need of such treatment a therapeutically effective amount of alectinib, or a pharmaceutically acceptable salt thereof, wherein the subject is aged <18 years.

### CROSS-REFERENCE TO RELATED APPLICATIONS

This application is a Continuation of International Patent Application No. PCT/EP2024/082092, filed Nov. 13, 2024, which claims benefit of priority to European Application Number 23210216.0 filed Nov. 15, 2023, each of which is incorporated herein by reference in its entirety.

### FIELD OF THE INVENTION

The present invention relates to method of treating an anaplastic lymphoma kinase (ALK) fusion-positive solid tumour or central nervous system tumour in paediatric subjects.

### BACKGROUND

Anaplastic lymphoma kinase (ALK) is a transmembrane receptor tyrosine kinase, which belongs to the insulin receptor superfamily. The ALK protein has an extracellular ligand-binding domain, a transmembrane region and an intracellular kinase domain. Ligand-dependent activation leads to dimerization and autophosphorylation resulting in the activation of the ALK pathway cascade and other signalling pathways, which affect cell growth, transformation, and anti-apoptotic signalling. Wild-type ALK is expressed at very low levels in most normal human tissues but is expressed at higher levels in a few limited types of tissue, such as developing and mature nervous system tissue.

Dysregulated ALK signalling results from various genetic aberrations, rearrangements, mutations, fusions, and amplification. Translocations in the ALK gene may lead to the production of constitutively activated ALK fusion proteins, which can activate downstream signalling pathways and lead to cell survival, proliferation and migration. The role of ALK fusion as a prominent initiator of carcinogenesis has been extensively demonstrated in in vivo, in vitro, and in clinical studies (Soda et al., 2007, Nature, 448:561-6; Inamura et al., 2008, Thorac Oncol, 3:13-7; Inamura et al., 20009, Mod Pathol, 22:508-15; Wong et al., 2009, Cancer, 15:1723-33; Clarke et al., 2020, Cancer Discov, 10(7):942-963).

ALK inhibition as a therapeutic intervention has been most extensively investigated in adult non-small cell lung cancer (NSCLC). Several studies evaluating ALK inhibitors in adult participants with metastatic or relapsed NSCLC demonstrated significant benefit in those participants, and ALK inhibition is now considered standard of care (SOC) in this disease segment (Cardoso et al. 2018, Ann Oncol, 29:1634-57; Ettinger et al., 2019, J Natl Compr Canc Netw, 17:1464-72).

Alectinib is a CNS penetrant molecule (Peters et al., 2017, N Engl J Med; 377:829-38) and is considered as the best in class ALK inhibitor for the first-line treatment of ALK fusion-positive NSCLC according to National Comprehensive Cancer Network (NCCN)® and European Society for Medical Oncology (ESMO) guidelines based on the outcome of the ALEX study (Camidge et al., 2019, J Thorac Oncol, 14:1233-43).

In the paediatric population, ALK fusions are identified in a variety of solid tumours, such as inflammatory myofibroblastic tumour (IMT) and bone and soft tissue sarcoma, and in primary CNS tumours, such as high-grade glioma (HGG). These fusions are described as oncogenic drivers (Mosse et al. 2016, Clin Cancer Res, 22:546-52; Ross et al. 2017, Oncologist, 22:1444-50; Takita, 2017, Cancer Sci, 108:1913-20). More than 50% of IMTs and 25% of HGGs are ALK fusion-positive (Guerreiro Stucklin et al., 2019, Nat Comm, 10:43; Casanova et al., 2020, Eur J Cancer, 127:123-9.; Clarke et al., 2020, Cancer Discov, 10(7):942-963). In paediatric participants, the outcome of relapsed, refractory, or metastatic tumours harbouring ALK fusions, remains poor.

Thus, there remains a significant unmet clinical need for paediatric patients with tumours harbouring ALK-fusions, including infantile high grade gliomas and inflammatory myofibroblastic tumours at diagnosis and relapse.

### SUMMARY OF THE INVENTION

The present inventors have found that alectinib is surprisingly effective at treating anaplastic lymphoma kinase (ALK) fusion-positive solid tumours or central nervous system tumours in the paediatric population.

The invention provides a method of treating an ALK fusion-positive solid tumour or central nervous system tumour, comprising administering to a subject in need of such treatment a therapeutically effective amount of alectinib, or a pharmaceutically acceptable salt thereof, wherein the subject is aged <18 years.

In some embodiments, the ALK fusion-positive central nervous system tumour is a non-metastatic or primary central nervous system tumour.

In some embodiments, the ALK fusion-positive solid tumour or central nervous system tumour is a neuroblastoma, renal cell carcinoma, papillary or medullary thyroid cancer, skin tumour, such as Spitz tumour or melanoma, inflammatory myofibroblastic tumour, soft tissue sarcoma, such as undifferentiated or rhabdomyosarcoma, osteosarcoma, malignant peripheral nerve sheath tumour, anaplastic large cell lymphoma, ganglioglioma, or glioma (low- or high-grade). Preferably, the ALK fusion-positive solid tumour or central nervous system tumour is an inflammatory myofibroblastic tumour, high-grade glioma, renal cell carcinoma, or anaplastic large cell lymphoma.

The ALK fusion-positive solid tumour or central nervous system tumour may comprise a mutation wherein ALK is fused to KIFC, TPM3, CLTC, PLEKHA7, DCTN1 or HNRNPA3.

In some embodiments, the subject is aged <2 years.

In some embodiments, the subject is aged <6 years.

In some embodiments, the subject is aged <12 years.

In some embodiments, the subject is aged ≥2 to <6 years.

In some embodiments, the subject is aged ≥6 to <12 years.

In some embodiments, the subject is aged ≥12 to <18 years.

In some embodiments, the subject does not comprise a mutation conferring resistance to ALK inhibitors.

In some embodiments, the subject has not had prior exposure to an ALK inhibitor.

In some embodiments, the subject has had prior treatment for an ALK fusion-positive solid tumour or central nervous system tumour. Thus, the subject may have a relapsed or refractory tumour.

The alectinib, or a pharmaceutically acceptable salt thereof, may be administered orally. The alectinib, or a pharmaceutically acceptable salt thereof, may be administered twice daily.

The total daily dose of alectinib, or a pharmaceutically acceptable salt thereof, may be between 50 and 1500 mg, preferably wherein:
i. the subject is ≥40 kg, and the total daily dose of alectinib, or pharmaceutically acceptable salt thereof, is between 600 and 1500 mg;
ii. the subject is ≥35 to <40 kg, and the total daily dose of alectinib, or pharmaceutically acceptable salt thereof, is between 400 and 1000 mg;
iii. the subject is ≥30 to <35 kg, and the total daily dose of alectinib, or pharmaceutically acceptable salt thereof, is between 300 and 800 mg;
iv. the subject is ≥25 to <30 kg, and the total daily dose of alectinib, or pharmaceutically acceptable salt thereof, is between 300 and 700 mg;
v. the subject is ≥20 to <25 kg, and the total daily dose of alectinib, or pharmaceutically acceptable salt thereof, is between 250 and 600 mg;
vi. the subject is ≥15 to <20 kg, and the total daily dose of alectinib, or pharmaceutically acceptable salt thereof, is between 200 and 500 mg;
vii. the subject is ≥10 to <15 kg, and the total daily dose of alectinib, or pharmaceutically acceptable salt thereof, is between 150 and 400 mg;
viii. the subject is ≥7.5 to <10 kg, and the total daily dose of alectinib, or pharmaceutically acceptable salt thereof, is between 100 and 300 mg;
ix. the subject is ≥5 to <7.5 kg, and the total daily dose of alectinib, or pharmaceutically acceptable salt thereof, is between 75 and 250 mg; or
x. the subject is ≥3.5 to <5 kg, and the total daily dose of alectinib, or pharmaceutically acceptable salt thereof, is between 50 and 200 mg.

In some embodiments, the alectinib, or a pharmaceutically acceptable salt thereof, is administered in a pharmaceutical composition. Preferably, the pharmaceutical composition comprises alectinib hydrochloride.

In some embodiments, the pharmaceutical composition is formulated as a capsule, preferably a hard capsule. The capsule may comprise alectinib hydrochloride equivalent to 20 mg or 150 mg alectinib as free base. In some embodiments, the pharmaceutical composition may be formulated as a suspension.

In some embodiments, the pharmaceutical composition is formulated as a capsule and:
i. the subject is ≥40 kg and the total daily dose of alectinib, or pharmaceutically acceptable salt thereof, is between 1000 and 1400 mg, preferably the total daily dose is 1200 mg, more preferably 600 mg BID;
ii. the subject is ≥35 to <40 kg and the total daily dose of alectinib, or pharmaceutically acceptable salt thereof, is between 700 and 900 mg, preferably the total daily dose is 760 mg, even more preferably 380 mg BID;
iii. the subject is ≥30 to <35 kg and the total daily dose of alectinib, or pharmaceutically acceptable salt thereof, is between 600 and 800 mg, preferably the total daily dose is 680 mg, even more preferably 340 mg BID;
iv. the subject is ≥25 to <30 kg and the total daily dose of alectinib, or pharmaceutically acceptable salt thereof, is between 500 and 700 mg, preferably the total daily dose is 600 mg, even more preferably 300 mg BID;
v. the subject is ≥20 to <25 kg and the total daily dose of alectinib, or pharmaceutically acceptable salt thereof, is between 500 and 600 mg, preferably the total daily dose is 540 mg, even more preferably 270 mg BID;
vi. the subject is ≥15 to <20 kg and the total daily dose of alectinib, or pharmaceutically acceptable salt thereof, is between 400 and 500 mg, preferably the total daily dose is 460 mg, even more preferably 230 mg BID;
vii. the subject is ≥10 to <15 kg and the total daily dose of alectinib, or pharmaceutically acceptable salt thereof, is between 300 and 400 mg, preferably the total daily dose is 380 mg, even more preferably 190 mg BID;
viii. the subject is ≥7.5 to <10 kg and the total daily dose of alectinib, or pharmaceutically acceptable salt thereof, is between 200 and 300 mg, preferably the total daily dose is 280 mg, even more preferably 140 mg BID;
ix. the subject is ≥5 to <7.5 kg and the total daily dose of alectinib, or pharmaceutically acceptable salt thereof, is between 150 and 250 mg total, preferably the total daily dose is 200 mg, even more preferably 100 mg BID; or
x. the subject is ≥3.5 to <5 kg and the total daily dose of alectinib, or pharmaceutically acceptable salt thereof, is between 100 and 200 mg, preferably the total daily dose is 160 mg, even more preferably 80 mg BID.

In some embodiments, the pharmaceutical composition is formulated as a capsule and:
i. the subject is ≥40 kg and the total daily dose of alectinib, or pharmaceutically acceptable salt thereof, is between 600 and 800 mg, preferably the total daily dose is 718 mg, even more preferably 359 mg BID;
ii. the subject is ≥35 to <40 kg and the total daily dose of alectinib, or pharmaceutically acceptable salt thereof, is between 400 and 500 mg, preferably the total daily dose is 456 mg, even more preferably 228 mg BID;
iii. the subject is ≥30 to <35 kg and the total daily dose of alectinib, or pharmaceutically acceptable salt thereof, is between 350 and 450 mg, preferably the total daily dose is 408 mg, even more preferably 204 mg BID;
iv. the subject is ≥25 to <30 kg and the total daily dose of alectinib, or pharmaceutically acceptable salt thereof, is between 300 and 400 mg, preferably the total daily dose is 360 mg, even more preferably 180 mg BID;
v. the subject is ≥20 to <25 kg and the total daily dose of alectinib, or pharmaceutically acceptable salt thereof, is between 280 and 380 mg, preferably the total daily dose is 324 mg, even more preferably 162 mg BID;
vi. the subject is ≥15 to <20 kg and the total daily dose of alectinib, or pharmaceutically acceptable salt thereof, is between 220 and 320 mg, preferably the total daily dose is 276 mg, even more preferably 138 mg BID;
vii. the subject is ≥10 to <15 kg and the total daily dose of alectinib, or pharmaceutically acceptable salt thereof, is between 180 and 280 mg, preferably the total daily dose is 228 mg, even more preferably 114 mg BID;
viii. the subject is ≥7.5 to <10 kg and the total daily dose of alectinib, or pharmaceutically acceptable salt thereof, is between 100 and 200 mg, preferably the total daily dose is 168 mg, even more preferably 84 mg BID;
ix. the subject is ≥5 to <7.5 kg and the total daily dose of alectinib, or pharmaceutically acceptable salt thereof, is between 80 and 180 mg, preferably the total daily dose is 120 mg total, even more preferably 60 mg BID; or
x. the subject is ≥3.5 to <5 kg and the total daily dose of alectinib, or pharmaceutically acceptable salt thereof, is between 50 and 150 mg, preferably the total daily dose is 96 mg, even more preferably 48 mg BID.

The invention provides alectinib, or a pharmaceutically acceptable salt thereof, for use in a method of treating an ALK fusion-positive solid tumour or central nervous system tumour in a subject, wherein the subject is aged <18 years.

The invention provides for use of a therapeutically effective amount of alectinib, or a pharmaceutically acceptable salt thereof, in the manufacture of a medicament for use in treating an ALK fusion-positive solid tumour or central nervous system tumour in a subject, wherein the subject is aged <18 years.

### DETAILED DESCRIPTION OF THE INVENTION

### Alectinib

Alectinib is a tetracyclic compound represented by the following formula (I):

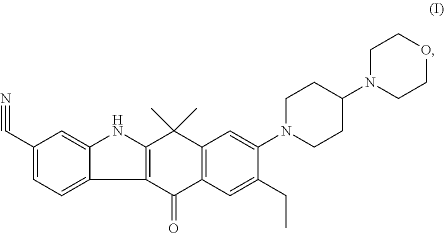 [SMILES: CCc1cc2c(cc1N1CCC(N3CCOCC3)CC1)C(C)(C)c1[nH]c3cc(C#N)ccc3c1C2=O]

having the compound name 9-ethyl-6,6-dimethyl-8-(4-morpholin-4-yl-piperidin-1-yl)-11-oxo-6,11-dihydro-5H-benzo[b]carbazole-3-carbonitrile (CAS Nos. 1256580-46-7 and 1256589-74-8 as hydrochloride).

Alectinib, and salts thereof, can be produced by methods commonly known in the art, for example, the general methodology described in WO 2010/143664 (see, in particular, Example 366, Compound “F6-20”). See also WO 2012/023597 (see, in particular, Production Example 30, Compound “F6-20”).

Alectinib is approved for use in the treatment of patients with anaplastic lymphoma kinase (ALK)-positive metastatic non-small cell lung cancer (NSCLC), as detected by an FDA-approved test. The approval covers patients with previously untreated metastatic lung cancer whose tumours have a cancer-causing alteration in the ALK gene (also called ALK positive). FDA previously approved alectinib as second-line therapy for patients with ALK-positive NSCLC whose tumours are resistant to crizotinib or who can no longer tolerate the treatment due to side effects.

As used herein, alectinib can be used in free base form, or alternatively can be in the form of a pharmaceutically acceptable salt.

Pharmaceutically acceptable salts include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Examples of preferred salts include hydrochloride, hydrobromide, hydroiodide, phosphate, phosphonate, sulfate, bisulphate, hemisulphate, sulfonate such as methanesulfonate and p-toluene sulfonate; carboxylate such as an acetate, trifluoroacetate, citrate, malate, maleate, tartrate, succinate and salicylate; alkali metal salts such as a sodium salt, a potassium salt; an alkaline earth metal salt such as a magnesium salt, a calcium salt; and ammonium salts such as an ammonium salt (NH4 X: X is a monovalent acid group), an alkylammonium salt, a dialkylammonium salt, a trialkylammonium salt and a tetraalkylammonium salt.

In one preferred embodiment, the alectinib is in the form of a hydrochloride salt, more preferably a monohydrochloride salt.

The present invention also includes solvate forms of alectinib. Preferably the solvate is a hydrate.

In one embodiment, the alectinib is in the form of a hydrate of a monohydrochloride salt, more preferably, a monohydrate.

The alectinib, or pharmaceutically acceptable salt thereof, may be in amorphous form or crystalline form.

The invention encompasses the use of various crystalline forms, polymorphic forms and (an)hydrous forms of alectinib. It is well established within the pharmaceutical industry that chemical compounds may be isolated in any of such forms by slightly varying the method of purification and or isolation form the solvents used in the synthetic preparation of such compounds.

In one embodiment, the alectinib is in amorphous form. An amorphous monohydrochloride form of alectinib can be produced in accordance with the teachings of WO 2016/021707.

In one embodiment, the alectinib is in crystalline form, preferably a crystalline form of a monohydrochloride salt. Suitable crystalline forms are known in the art, and are taught, for example, in WO 2015/163448 and WO 2015/163447.

The invention includes all enantiomers and tautomers of alecitnib and pharmaceutically acceptable salts thereof. The person skilled in the art will recognise that alecitnib possesses a chiral carbon atom. The corresponding enantiomers and/or tautomers may be isolated/prepared by methods known in the art. Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985). Compounds containing a chiral centre may be used as a racemic mixture, an enantiomerically enriched mixture, or the racemic mixture may be separated using well-known techniques and an individual enantiomer may be used alone.

### Pharmaceutical Compositions

For use according to the present invention, the alectinib, or pharmaceutically acceptable salt thereof, is generally formulated with one or more pharmaceutically acceptable carriers, diluents or excipients therefor. The carrier(s) must be acceptable in the sense of being compatible with the other ingredients of the formulation and not deleterious to the recipient thereof.

Examples of suitable excipients for the various different forms of pharmaceutical compositions described herein may be found in the “Handbook of Pharmaceutical Excipients, 2^{nd} Edition, (1994), Edited by A Wade and PJ Weller. The carrier, or, if more than one be present, each of the carriers, must be acceptable in the sense of being compatible with the other ingredients of the formulation and not deleterious to the recipient.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including antioxidants) and the like, disintegrating agents, dissolution aids, and substances included for the purpose of rendering the formulation isotonic with the blood of the intended recipient.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Examples of suitable binders include hydroxypropylcellulose, hydroxypropylmethylcellulose, carboxymethyl cellulose methylcellulose, povidone (polyvinylpyrrolidone), macrogol, powdered acacia, starch, gelatin, natural sugars such as glucose, anhydrous lactose, free-flow lactose, beta-lactose, corn sweeteners, natural and synthetic gums, such as acacia, tragacanth or sodium alginate, and polyethylene glycol. A preferred binder is hydroxypropylcellulose.

Examples of suitable lubricants include magnesium stearate, calcium stearate, talc, sucrose fatty acid ester and sodium stearyl fumarate. Preferably, magnesium stearate may be mentioned.

Examples of suitable stabilizers include paraoxybenzoates such as methyl paraben and propyl paraben; alcohols such as chlorobutanol, benzyl alcohol and phenylethyl alcohol; benzalkonium chloride, phenols such as phenol and cresol, thimerosal, dehydroacetic acid and sorbic acid.

Examples of suitable flavoring agents include a sweetener, an acidulant and a fragrance.

Examples of suitable disintegrating agents include sodium starch glycolate, low-substituted hydroxypropylcellulose, carboxymethylcellulose calcium (carmellose calcium), sodium hydrogen carbonate, pregelatinized starch, sodium chloride, corn starch, croscarmellose sodium, crystalline cellulose, silicic anhydride, carmellose, etc.

Examples of suitable dissolution aids include a surfactant, an organic polymer, and a pH adjusting agent, etc. Preferred examples thereof include casein, sodium caseinate, skimmed milk powder, sodium lauryl sulfate (herein below, also referred to as SLS), dioctyl sodium sulfosuccinate, sorbitan trioleate, polyoxyethylene (105) polyoxypropylene (5) glycol, polyoxyethylene hydrogenated castor oil 60, polyoxyl 35 castor oil, sodium lauroylsarcosine, sodium tetradecyl sulfate, sodium hexadecyl sulfate, sodium octadecyl sulfate, sodium methyl sulfate, sodium ethyl sulfate, sodium butyl sulfate, sodium octyl sulfate, sodium decyl sulfate, and sodium dodecylbenzene sulfonate.

Suitable solubilizing agents include surfactants, organic polymers and pH modifiers.

Examples of surfactants include ionic surfactants and nonionic surfactants. Ionic surfactants are further classified into anionic surfactants, cationic surfactants and amphoteric surfactants depending upon the charge of the ion to be produced. Examples of nonionic surfactants include sugar ester surfactants such as a sorbitan fatty acid ester (C12-18), a POE sorbitan fatty acid ester (C12-18) and sucrose fatty acid ester; fatty acid ester surfactants such as a POE fatty acid ester (C12-18), a POE resin acid ester and a POE fatty acid diester (C12-18); alcohol based surfactants such as a POE alkyl ether (C12-18); alkyl phenol surfactants such as a POE alkyl (C8-12) phenyl ether, a POE dialkyl (C8-12) phenyl ether and a POE alkyl (C8-12) phenyl ether formalin condensate; polyoxyethylene-polyoxypropylene block polymer surfactants such as a polyoxyethylene-polyoxypropylene block polymer and an alkyl (C12-18) polyoxyethylene-polyoxypropylene block polymer ether; alkylamine surfactants such as a POE alkylamine (C12-18) and a POE fatty acid amide (C12-18); bisphenol surfactants such as a POE fatty acid bisphenyl ether; polycyclic aromatic surfactants such as a POA benzylphenyl (or phenylphenyl) ether and a POA styrylphenyl (or phenylphenyl) ether; POE ether and ester type silicon and fluorine surfactants; and vegetable oil surfactants such as POE castor oil and POE hydrogenated castor oil.

Examples of anionic surfactants include sulfate surfactants such as an alkyl sulfate (C12-18, Na, NH4, alkanolamine), a POE alkyl ether sulfate (C12-18, Na, NH4, alkanolamine), a POE alkyl phenyl ether sulfate (C12-18, NH4, alkanolamine, Ca), a POE benzyl (or styryl) phenyl (or phenylphenyl) ether sulfate (Na, NH4, alkanolamine), a polyoxyethylene and a polyoxypropylene block polymer sulfate (Na, NH4, alkanolamine); sulfonate surfactants such as a paraffin (alkane) sulfonate (C12-22, Na, Ca, alkanolamine), an AOS (C14-16, Na, alkanolamine), a dialkyl sulfosuccinate (C8-12, Na, Ca, Mg), an alkylbenzene sulfonate (C12, Na, Ca, Mg, NH4, alkylamine, alkanol, amine, cyclohexylamine), a mono or dialkyl (C₃-6) naphthalene sulfonate (Na, NH4, alkanolamine, Ca, Mg), a naphthalene sulfonate-formalin condensate (Na, NH4), an alkyl (C8-12) diphenyl ether disulfonate (Na, NH4), a lignin sulfonate (Na, Ca), a POE alkyl (C8-12) phenyl ether sulfonate (Na) and a POE alkyl (C12-18) ether sulfosuccinic acid half ester (Na); carboxylic acid surfactants such as a fatty acid salt (C12-18, Na, K, NH4, alkanolamine), a N-methyl-fatty acid sarcosinate (C12-18, Na) and a resinate (Na, K); and phosphate surfactants such as a POE alkyl (C12-18) ether phosphate (Na, alkanolamine), a POE mono or dialkyl (C8-12) phenyl ether phosphate (Na, alkanolamine), a POE benzylated (or styrylated) phenyl (or phenylphenyl) ether phosphate (Na, alkanolamine), a polyoxyethylene-polyoxypropylene block polymer (Na, alkanolamine), a phosphatidylcholine-phosphatidyl ethanol imine (lecithin) and an alkyl (C8-12) phosphate. Preferably, e.g., monoalkyl sulfates such as sodium lauryl sulfate, sodium tetradecyl sulfate, sodium hexadecyl sulfate and sodium octadecyl sulfate, dioctylsodium sulfosuccinate, sodium lauroyl sarcosinate and sodium dodecylbenzenesulfonate are mentioned.

Sodium lauryl sulfate is a particularly preferred surfactant. In the present invention, when sodium lauryl sulfate is used, it is more preferable to obtain a crystal thereof by crystallization rather than spray dry. As a crystal polymorphism of sodium lauryl sulfate, a monohydrate, a ½ hydrate, a ⅛ hydrate and a non-solvate are known (Journal of Crystal Growth 263 (2004) 480-490). Any of the crystals can be used.

In one embodiment, the alectinib or pharmaceutically acceptable salt thereof is formulated in accordance with the teachings of WO 2012/023597. For example, in one preferred embodiment the composition comprises alectinib, or a pharmaceutically acceptable salt thereof, a pharmaceutically acceptable carrier, and a dissolution aid.

In another embodiment, alectininib, or pharmaceutically acceptable salt thereof, is formulated in accordance with the teachings of WO 2015/163448. For example, in one preferred embodiment, the pharmaceutical composition comprises (i) a granule containing alectinib, or a pharmaceutically acceptable salt thereof, and (ii) a disintegrating agent.

The “disintegrating agent” is a component facilitating rapid disintegration of a solid formulation taken orally. Suitable examples are described above. The disintegrating agent can be contained in the granules or added to the granules as an external additive. Other additives such as a lubricant and a fluidizer may be further optionally added as external additives, or contained in the granules. In one embodiment, the granule (i) contains alectinib, or a pharmaceutically acceptable salt thereof, a disintegrating agent, a solubilizing agent, an excipient and a binder. The granule may further contain one or more additives selected from a lubricant, a coating agent, a stabilizer, a flavoring agent and a diluent.

In one preferred embodiment, the alectinib, or pharmaceutically acceptable salt thereof, is formulated for oral administration, i.e. the composition is an oral formulation.

In one embodiment, the composition is formulated as a tablet. An appropriate coating agent can be further applied to tablets to obtain sugar coated tablets or film coated tablets. Suitable coating agents will be familiar to the person skilled in the art.

In one preferred embodiment, the pharmaceutical composition comprises alectinib, or a pharmaceutically acceptable salt thereof, lactose monohydrate, hydroxypropylcellulose, sodium lauryl sulfate, magnesium stearate and carmellose calcium.

Preferably the pharmaceutical composition is in the form of a capsule, more preferably a hard capsule.

Preferably the composition is provided in a capsule shell comprising hypromellose, carrageenan, potassium chloride, titanium dioxide (E171), maize starch and carnauba wax.

In one embodiment, the pharmaceutical composition is in the form of a suspension.

A correction factor of 1.67 may be applied to an intact capsule dose in order to obtain an oral suspension dose that ensures a similar exposure.

The formulation of the present invention preferably contains alectinib, or a pharmaceutically acceptable salt thereof, in an amount of, for example, about 20 to about 70 wt %, preferably, about 30 to about 60 wt %, and especially preferably, about 35 to about 60 wt % in terms of free base alectinib based on the total amount of the formulation.

More specifically, if the formulation of the present invention is a capsule, the alectinib or pharmaceutically acceptable salt thereof is preferably contained in an amount of, for example, about 20 to about 70 wt %, preferably, about 30 to about 60 wt %, and especially preferably, about 35 to about 60 wt % in terms of free base alectinib based on the total amount of the components to be put in a capsule.

In one preferred embodiment, the composition is in the form of a unit dosage form comprising alectinib or a pharmaceutically acceptable salt thereof in an amount of, for example, 20 to 240 mg, preferably, 100 to 200 mg, and especially preferably, 140 mg to 190 mg in terms of free base alectinib per unit dosage form.

In one preferred embodiment, the composition is in the form of a unit dosage form comprising alectinib hydrochloride equivalent to about 150 mg alectinib in free base form.

In one preferred embodiment, the formulation is in a dosage form comprising alectinib hydrochloride equivalent to about 150 mg alectinib in free base form, about 33.7 mg lactose (as monohydrate) and about 6 mg sodium (as sodium lauryl sulfate) per dosage form.

In one preferred embodiment, the composition is in the form of a unit dosage form comprising alectinib hydrochloride equivalent to about 20 mg alectinib in free base form.

The pharmaceutical compositions as described above can be prepared by a process which comprises bringing the active compound into association with the carrier, for example by admixture.

The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

### Dosage

The composition may be administered orally, rectally, parenterally (intravenously, intramuscularly, subcutaneously), intracisternally, intravaginally, intraperitoneally, intravesically and locally (intravenous drip, powder, ointment, gel or cream) and through inhalation (oral or nose spray). Examples of the dosage form thereof include tablet, capsule, granule, powder, pill, aqueous and nonaqueous oral solution and suspension, and parenteral solution put in small containers for a unit dose. The dosage form may be designed so as to adapt to various methods for administrating a formulation including a controlled release formulation to be subcutaneously grafted.

The dosage form is preferably for oral administration by tablets, capsules, granules, suspension or powder formulation.

Preferably, the dosage form is a capsule or oral suspension.

In the present invention, the “oral formulation” refers to a formulation that can be orally administered. The oral administration refers to swallowing a formulation so as to enter the gastrointestinal tract and an active ingredient is mainly absorbed through the intestinal tract.

Specific examples of the oral formulation include solid formulations such as a tablet, a capsule, a solution, a powder, a lozenge, a chewable agent, a granule, a gel, a film agent and a spray and liquid formulations. Examples of the liquid formulation include a suspension, a solution, syrup and an elixir. Such a formulation can be used as filler in soft or hard capsules. Generally, as a carrier, for example, water, ethanol, polyethylene glycol, propylene glycol, methylcellulose, or appropriate oil, and one or more emulsifiers and/or a suspending agent are used. A liquid formulation can be also prepared by dissolving a unit-dose solid medicinal agent, for example, dissolving a pharmaceutical composition in a package into a carrier such as water.

The dosage of the active ingredient (a compound represented by formula (I) or a salt thereof of the present invention) may vary depending upon the symptom, age, body weight, relative health condition, other medication, administering method, etc. For example, a generally effective amount of active ingredient (the compound or a salt thereof of the present invention represented by formula (I)) to a patient (a warm-blooded animal, particularly a human) orally administered is preferably 0.001 to 1000 mg and further preferably 0.01 to 300 mg per body weight (1 kg) per day. The dosage thereof used per day preferably falls within the range of 1 to 1500 mg per adult patient having an ordinary body weight. In the case of a parenteral agent, the amount thereof used is preferably 0.001 to 1000 mg and further preferably 0.01 to 300 mg per body weight (1 kg) per day. It is desirable that the amount thereof used is administered once per day or administered depending upon the symptom by dividing the amount used into several portions.

In one embodiment, the alectinib, or pharmaceutically acceptable salt thereof, is administered twice a day.

The total daily dose of alectinib (free base equivalent) is preferably from 1 to 1500 mg, 100 to 1400 mg, 200 to 1300 mg, 400 to 1200 mg, 600 to 1500 mg, 700 to 1500 mg, 800 to 1500 mg, 900 to 1500 mg, 600 to 1200 mg, 700 to 1200 mg, 800 to 1200 mg, 900 to 1200 mg, 1000 to 1200 mg.

The total daily dose of alectinib may be adjusted based on the body weight of the subject.

In one embodiment, the subject is ≥40 kg and the total daily dose of alectinib, or pharmaceutically acceptable salt thereof, is between 600 and 1500 mg.

In one embodiment, the subject is ≥35 to <40 kg and the total daily dose of alectinib, or pharmaceutically acceptable salt thereof, is between 400 and 1000 mg.

In one embodiment, the subject is ≥30 to <35 kg and the total daily dose of alectinib, or pharmaceutically acceptable salt thereof, is between 300 and 800 mg.

In one embodiment, the subject is ≥25 to <30 kg and the total daily dose of alectinib, or pharmaceutically acceptable salt thereof, is between 300 and 700 mg.

In one embodiment, the subject is ≥20 to <25 kg and the total daily dose of alectinib, or pharmaceutically acceptable salt thereof, is between 250 and 600 mg.

In one embodiment, the subject is ≥15 to <20 kg and the total daily dose of alectinib, or pharmaceutically acceptable salt thereof, is between 200 and 500 mg.

In one embodiment, the subject is ≥10 to <15 kg and the total daily dose of alectinib, or pharmaceutically acceptable salt thereof, is between 150 and 400 mg.

In one embodiment, the subject is ≥7.5 to <10 kg and the total daily dose of alectinib, or pharmaceutically acceptable salt thereof, is between 100 and 300 mg.

In one embodiment, the subject is ≥5 to <7.5 kg and the total daily dose of alectinib, or pharmaceutically acceptable salt thereof, is between 75 and 250 mg.

In one embodiment, the subject is ≥3.5 to <5 kg and the total daily dose of alectinib, or pharmaceutically acceptable salt thereof, is between 50 and 200 mg.

The total daily dose of alectinib may be adjusted based on the formulation of the pharmaceutical composition. For example, a correction factor of 1.67 may be applied to an intact capsule dose in order to obtain an oral suspension dose that ensures a similar exposure.

In one embodiment, the pharmaceutical composition is formulated as a capsule and the subject is ≥40 kg, and the total daily dose of alectinib, or pharmaceutically acceptable salt thereof, is between 1000 and 1400 mg, preferably 1200 mg total, even more preferably 600 mg BID.

In one embodiment, the pharmaceutical composition is formulated as a capsule and the subject is ≥35 to <40 kg, and the total daily dose of alectinib, or pharmaceutically acceptable salt thereof, is between 700 and 900 mg, preferably 760 mg total, even more preferably 380 mg BID.

In one embodiment, the pharmaceutical composition is formulated as a capsule and the subject is ≥30 to <35 kg, and the total daily dose of alectinib, or pharmaceutically acceptable salt thereof, is between 600 and 800 mg, preferably 680 mg total, even more preferably 340 mg BID.

In one embodiment, the pharmaceutical composition is formulated as a capsule and the subject is ≥25 to <30 kg, and the total daily dose of alectinib, or pharmaceutically acceptable salt thereof, is between 500 and 700 mg, preferably 600 mg total, even more preferably 300 mg BID.

In one embodiment, the pharmaceutical composition is formulated as a capsule and the subject is ≥20 to <25 kg, and the total daily dose of alectinib, or pharmaceutically acceptable salt thereof, is between 500 and 600 mg, preferably 540 mg total, even more preferably 270 mg BID.

In one embodiment, the pharmaceutical composition is formulated as a capsule and the subject is ≥15 to <20 kg, and the total daily dose of alectinib, or pharmaceutically acceptable salt thereof, is between 400 and 500 mg, preferably 460 mg total, even more preferably 230 mg BID.

In one embodiment, the pharmaceutical composition is formulated as a capsule and the subject is ≥10 to <15 kg, and the total daily dose of alectinib, or pharmaceutically acceptable salt thereof, is between 300 and 400 mg, preferably 380 mg total, even more preferably 190 mg BID.

In one embodiment, the pharmaceutical composition is formulated as a capsule and the subject is ≥7.5 to <10 kg, and the total daily dose of alectinib, or pharmaceutically acceptable salt thereof, is between 200 and 300 mg, preferably 280 mg total, even more preferably 140 mg BID.

In one embodiment, the pharmaceutical composition is formulated as a capsule and the subject is ≥5 to <7.5 kg, and the total daily dose of alectinib, or pharmaceutically acceptable salt thereof, is between 150 and 250 mg, preferably 200 mg total, even more preferably 100 mg BID.

In one embodiment, the pharmaceutical composition is formulated as a capsule and the subject is ≥3.5 to <5 kg, and the total daily dose of alectinib, or pharmaceutically acceptable salt thereof, is between 100 and 200 mg, preferably 160 mg total, even more preferably 80 mg BID.

In one embodiment, the pharmaceutical composition is formulated as a suspension and the subject is ≥40 kg, and the total daily dose of alectinib, or pharmaceutically acceptable salt thereof, is between 600 and 800 mg, preferably 718 mg total, even more preferably 359 mg BID.

In one embodiment, the pharmaceutical composition is formulated as a suspension and the subject is ≥35 to <40 kg, and the total daily dose of alectinib, or pharmaceutically acceptable salt thereof, is between 400 and 500 mg, preferably 456 mg total, even more preferably 228 mg BID.

In one embodiment, the pharmaceutical composition is formulated as a suspension and the subject is ≥30 to <35 kg, and the total daily dose of alectinib, or pharmaceutically acceptable salt thereof, is between 350 and 450 mg, preferably 408 mg total, even more preferably 204 mg BID.

In one embodiment, the pharmaceutical composition is formulated as a suspension and the subject is ≥25 to <30 kg, and the total daily dose of alectinib, or pharmaceutically acceptable salt thereof, is between 300 and 400 mg, preferably 360 mg total, even more preferably 180 mg BID.

In one embodiment, the pharmaceutical composition is formulated as a suspension and the subject is ≥20 to <25 kg, and the total daily dose of alectinib, or pharmaceutically acceptable salt thereof, is between 280 and 380 mg preferably 324 mg total, even more preferably 162 mg BID.

In one embodiment, the pharmaceutical composition is formulated as a suspension and the subject is ≥15 to <20 kg, and the total daily dose of alectinib, or pharmaceutically acceptable salt thereof, is between 220 and 320 mg, preferably 276 mg total, even more preferably 138 mg BID.

In one embodiment, the pharmaceutical composition is formulated as a suspension and the subject is ≥10 to <15 kg, and the total daily dose of alectinib, or pharmaceutically acceptable salt thereof, is between 180 and 280 mg, preferably 228 mg total, even more preferably 114 mg BID.

In one embodiment, the pharmaceutical composition is formulated as a suspension and the subject is ≥7.5 to <10 kg, and the total daily dose of alectinib, or pharmaceutically acceptable salt thereof, is between 100 and 200 mg, preferably 168 mg total, even more preferably 84 mg BID.

In one embodiment, the pharmaceutical composition is formulated as a suspension and the subject is ≥5 to <7.5 kg, and the total daily dose of alectinib, or pharmaceutically acceptable salt thereof, is between 80 and 180 mg, preferably 120 mg total, even more preferably 60 mg BID.

In one embodiment, the pharmaceutical composition is formulated as a suspension and the subject is ≥3.5 to <5 kg, and the total daily dose of alectinib, or pharmaceutically acceptable salt thereof, is between 50 and 150 mg, preferably 96 mg total, even more preferably 48 mg BID.

In a further embodiment the alectinib dosage can be reduced in increments in order to mitigate side effects. Such side effects can include any of the following or a combination thereof: pneumonitis, pneumonia, appendicitis, acute myocardial infarction, nausea, malaise, asthenia, fatigue, tinnitus, vomiting, weight increase, decreased appetite, diarrhoea, constipation, headache, cough, rash, anaemia, dyspnoea, oedema peripheral, dysgeusia, blood creatinine increase, neutropenia, neutrophil count decrease, white blood cell count decrease, blood creatine phosphokinase increase, aspartate aminotransferase increase, alanine aminotransferase increase, hyperbilirubinaemia, COVID-19, myalgia, blood alkaline phosphatase increase, and/or product dose omission issue.

In some embodiments, the dose may be reduced by approximately 25% of the starting dose. For example, a starting dose of 600 mg BID may be reduced to 450 mg BID.

In some embodiments, the dose may be further reduced by approximately 33%. For example, a total daily dose of 600 mg BID may be initially reduced to 450 mg BID, and may be further reduced to 300 mg BID.

Treatment may continue until completion of treatment period, disease recurrence, unacceptable toxicity, withdrawal of consent, or death. The treatment period may be 1, 2, 3, 4, 5, 6, 7, 8, 9, 10, 11, 12, 13, 14, 15, 16, 17, 17, 19, 20, 21, 22, 23, 24, 36, 48, or 60 months. In one embodiment, the treatment period is 6 months or 12 months or 18 months or 24 months.

### Disease

The term “tumour” as used herein, refers to all neoplastic cell growth and proliferation, whether malignant or benign, and all pre-cancerous and cancerous cells and tissues. The terms “cancer”, “cancerous” and “tumour” are not mutually exclusive as referred to herein.

The terms “cancer” and “cancerous” refer to or describe the physiological condition in mammals that is typically characterized by unregulated cell growth. Included in this definition are benign and malignant cancers.

By “early stage cancer” or “early stage tumour” is meant a cancer that is not invasive or metastatic or is classified as a Stage 0, I, or II cancer.

Examples of cancer include, but are not limited to, carcinoma, lymphoma, blastoma (including medulloblastoma and retinoblastoma), sarcoma (including liposarcoma and synovial cell sarcoma), neuroendocrine tumours (including carcinoid tumours, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, Merkel cell cancer, mycoses fungoids, testicular cancer, esophageal cancer, tumours of the biliary tract, as well as head and neck cancer and hematological malignancies.

NSCLC, includes squamous NSCLC and non-squamous NSCLC, adenocarcinoma, and large cell carcinoma.

In some embodiments, the cancer is ALK-positive.

By “ALK-positive” or “ALK+” is meant that the cancer expresses anaplastic lymphoma kinase.

In some embodiments, expression is caused by gene rearrangements leading to fusion of ALK with other genes, such as EML4, KIF5B, KLC1, TFG, TPR, HIP1, STRN, DCTN1, SQSTM1, NPM1, BCL11A, and BIRC6.

Thus, in some embodiments, the cancer is an anaplastic lymphoma kinase (ALK) fusion-positive solid tumour or central nervous system tumour.

Determination of ALK positivity may be performed at sites by an FDA-approved and CE-marked test or by central Ventana ALK IHC.

Methods to determine ALK positivity (e.g., reverse transcription-PCR) are known. ALK positivity for fusions may be determined in tissue samples using assays such as fluorescence in situ hybridization, IHC, PCR, and sequencing. As tissue sampling is difficult and tissue biopsy sample sizes from cancer patients are very small, testing of several important biomarkers is challenging. Plasma ALK assays analysing circulating tumour nucleic acids will enable more patients with cancer to be tested for ALK fusions and may also be used to monitor changes in ALK rearrangements during ALK inhibitor treatment.

In some embodiments, the Ventana ALK (D5F3) CDx Assay (Ventana Medical Systems, Tucson, AZ, USA) is used to determine ALK positivity.

In some embodiments, the subject does not demonstrate resistance to ALK inhibitors.

In some embodiments, the subject does not comprise a mutation conferring resistance to ALK inhibitors.

In some embodiments, the subject does not exhibit a biomarker associated with resistance to ALK inhibitors.

Molecular mechanisms (including mutations and associated biomarkers) of resistance to ALK inhibitors may include: secondary mutations in the ALK gene (e.g., gatekeeper mutation), increased copy number of the ALK gene, increased expression of ALK mRNA, and ALK-independent resistance mechanisms through activation of other oncogenic genes and pathways such as EGFR, cKIT, MET or KRAS.

In some embodiment, the ALK fusion-positive tumour is an ALK fusion-positive solid tumour. In some embodiment, the ALK fusion-positive tumour is an ALK fusion-positive CNS tumour. In some embodiments, the ALK fusion-positive solid tumour or central nervous system tumour is a neuroblastoma, renal cell carcinoma, papillary or medullary thyroid cancer, skin tumour, such as Spitz tumour or melanoma, inflammatory myofibroblastic tumour, soft tissue sarcoma, such as undifferentiated or rhabdomyosarcoma, osteosarcoma, malignant peripheral nerve sheath tumour, anaplastic large cell lymphoma, ganglioglioma, or glioma (low- or high-grade).

In some embodiments, the ALK fusion-positive solid tumour or central nervous system tumour is an inflammatory myofibroblastic tumour, high-grade glioma, renal cell carcinoma, or anaplastic large cell lymphoma.

In some embodiments, the ALK fusion-positive solid tumour or central nervous system tumour is an inflammatory myofibroblastic tumour or high-grade glioma.

In some embodiments, the ALK fusion-positive solid tumour or central nervous system tumour comprises a mutation wherein ALK is fused to KIFC, TPM3, CLTC, PLEKHA7, DCTN1 or HNRNPA3.

In some embodiments, the ALK fusion-positive solid tumour is a non-metastatic (i.e. primary) tumour. In some embodiments, the ALK fusion-positive solid tumour is a metastatic tumour.

In some embodiments, the ALK fusion-positive central nervous system tumour is a non-metastatic (i.e. primary) or metastatic tumour. Preferably, the ALK fusion-positive central nervous system tumour is a non-metastatic tumour.

In some embodiments, the subject has a histologically confirmed diagnosis of CNS or solid tumours harbouring ALK gene fusions as determined locally by an appropriately validated assay performed in a Clinical Laboratory Improvement Amendments (CLIA)-certified or equivalently-accredited diagnostic laboratory.

As used herein, “histologically confirmed” refers to determination by microscopic examination of tissue that has been excised by biopsy or surgical resection.

### Adjuvant

In one preferred embodiment, the alectinib, or pharmaceutically acceptable salt thereof, is administered as adjuvant therapy.

As used herein, “adjuvant” and/or “adjuvant setting” refers to a therapy given in addition to the primary or initial therapy, for example following resection of a tumour.

As used herein, “resection” refers to the surgical removal of all or part of a tumour. The tumour may be removed with a surrounding amount of normal, healthy tissue (surgical margin). Preferably, the subject has a negative surgical margin.

In some embodiments, resection is by lobectomy, sleeve lobectomy, bilobectomy, or pneumonectomy. Preferably, resection is not by segmentectomy or wedge resection.

In some embodiments, the subject has not undergone prior adjuvant radiotherapy. In some embodiments, the subject has not undergone prior adjuvant therapy.

Preferably, alectinib is administered as adjuvant therapy following complete resection of a tumour.

In some embodiments, alectinib is administered as adjuvant treatment following tumour resection in patients with ALK+ tumours as detected by an FDA-approved test.

As used herein, “neo-adjuvant” refers to treatment delivered before resection of tumour, such as to help reduce the size of a tumour.

Preferably, alectinib is not administered in the neo-adjuvant setting. Preferably, the subject has not been administered alectinib prior to resection of a tumour.

In some embodiments, the subject has not had prior exposure to ALK inhibitors.

In some embodiments, the subject has received radiotherapy prior to resection of a tumour.

In some embodiments, resection occurs between 1 to 16 weeks prior to treatment with alectinib, such as 4 to 12 weeks, 6 to 10 weeks, 4 to 6 weeks, 6 to 8 weeks, or 8 to 12 weeks prior to treatment with alectinib. For example, resection may occur at 1, 2, 3, 4, 5, 6, 7, 8, 9, 10, 11, 12, 13, 14, 15, or 16 weeks prior to treatment with alectinib.

Preferably, resection occurs between 4 to 12 weeks prior to treatment with alectinib.

### Method of Treatment

The invention provides a method of treating a subject suffering from an ALK fusion-positive solid tumour or central nervous system tumour, the method comprising administering to the subject a therapeutically effective amount of alectinib, wherein the subject is aged <18 years.

The invention provides methods for treating or delaying progression of an ALK fusion-positive solid tumour or central nervous system tumour in a subject comprising administering to the subject a therapeutically effective amount of alectinib, wherein the subject is aged <18 years.

The invention provides alectinib, or a pharmaceutically acceptable salt thereof, for use in a method of treating an ALK fusion-positive solid tumour or central nervous system tumour in a subject, wherein the subject is aged <18 years.

The invention provides alectinib, or a pharmaceutically acceptable salt thereof, for use in a method of treating or delaying progression of an ALK fusion-positive solid tumour or central nervous system tumour in a subject, wherein the subject is aged <18 years.

The invention provides a method of preventing ALK fusion-positive solid tumour or central nervous system tumour recurrence in a subject, comprising administering to a subject a therapeutically effective amount of alectinib, or a pharmaceutically acceptable salt thereof, wherein the subject is aged <18 years.

The invention provides alectinib, or a pharmaceutically acceptable salt thereof, for use in a method of preventing ALK-positive solid tumour or central nervous system tumour recurrence in a subject, wherein the subject is aged <18 years.

In some embodiments, the alectinib is administered as adjuvant therapy following complete resection of tumour. In the context of the present invention, resection refers to a surgery taking place before alectinib treatment. The present invention does not relate to methods of surgery performed on the human body.

In some embodiments, alectinib is for use in the treatment of ALK fusion-positive solid or CNS tumours in a subject for whom prior treatment has proven to be ineffective (i.e., relapsed or refractory), or for whom there is no satisfactory treatment, wherein the subject is aged <18 years.

Thus, in some embodiments, the subject has had prior treatment for an ALK fusion-positive solid tumour or central nervous system tumour.

As used herein, the terms “patient” or “subject” are used interchangeably and refer to any single animal, more preferably a mammal (including such non-human animals as, for example, dogs, cats, horses, rabbits, zoo animals, cows, pigs, sheep, and non-human primates) for which treatment is desired. In particular embodiments, the patient herein is a human.

In some embodiments, the subject is pediatric.

In some embodiments, the subject is adolescent.

In some embodiments, the subject is aged less than 18 years.

In some embodiments, the subject is aged less than 12 years.

In some embodiments, the subject is aged less than 6 years.

In some embodiments, the subject is aged less than 2 years.

In some embodiments, the subject is aged ≥2 to <6 years.

In some embodiments, the subject is aged ≥6 to <12 years.

In some embodiments, the subject is aged ≥12 to <18 years.

In some embodiments, the subject is aged <18 years, such as <2 years, ≥2 to <6 years, ≥6 to <12 years or ≥12 to <18 years, at the start of treatment.

As used herein, “treatment” refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions of the present invention are used to delay development of a disease or to slow the progression of a disease.

“Individual response” or “response” can be assessed using any endpoint indicating a benefit to the individual, including, without limitation, (1) inhibition, to some extent, of disease progression (e.g., cancer progression), including slowing down and complete arrest; (2) a reduction in tumour size; (3) inhibition (i.e., reduction, slowing down or complete stopping) of cancer cell infiltration into adjacent peripheral organs and/or tissues; (4) inhibition (i.e. reduction, slowing down or complete stopping) of metatasis; (5) relief, to some extent, of one or more symptoms associated with the disease or disorder (e.g., cancer); (6) increase or extend in the length of survival, including overall survival, progression free survival and disease free survival; and/or (9) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival, progression-free survival and disease-free survival); resulting in an objective response (including a complete response or a partial response); or improving signs or symptoms of cancer.

An “objective response” refers to a measurable response, including complete response (CR) or partial response (PR).

In some embodiments, the “objective response rate (ORR)” refers to the proportion of patients with a complete response (CR) or partial response (PR) on two consecutive occasions ≥4 weeks apart, as determined by the blinded independent central review (BICR) according to Response Assessment in Neuro-Oncology (RANO) for patients with primary CNS tumors, RECIST v1.1 for patients with extracranial tumors, and International Neuroblastoma Response Criteria (INRC) for patients with neuroblastoma.

As used herein, “partial response” or “PR” refers to a decrease in the size of one or more tumours or lesions, or in the extent of cancer in the body, in response to treatment and is defined according to RECIST v1.1, INRC or RANO criteria set out below (see Examples). For example, in some embodiments, PR refers to at least a 30% decrease in the sum of the longest diameters (SLD) of target lesions, taking as reference the baseline SLD.

By “complete response” or “CR” is intended the disappearance of all signs of cancer (e.g., disappearance of all target lesions) in response to treatment and is defined according to RECIST v1.1, INRC or RANO criteria set out below (see Examples). This does not always mean the cancer has been cured.

“Sustained response” refers to the sustained effect on reducing tumour growth after cessation of a treatment. For example, the tumour size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration, or longer. As used herein, “reducing or inhibiting cancer relapse” means to reduce or inhibit tumour or cancer relapse or tumour or cancer progression. As disclosed herein, cancer relapse and/or cancer progression include, without limitation, cancer metastasis.

As used herein, “stable disease” or “SD” refers to neither sufficient shrinkage of target lesions to qualify for PR, nor sufficient increase to qualify for PD, taking as reference the smallest SLD since the treatment started.

As used herein, “duration of response” (DOR) is defined as the time from the first occurrence of a documented objective response (either a CR or a PR) to disease progression or death from any cause (whichever occurs first), as determined by BICR and investigator according to RANO for patients with primary CNS tumours, RECIST v1.1 for extracranial tumors, and INRC for neuroblastoma.

As used herein, “time to response” (TTR) is defined as the time from the first dose of alectinib to the first documentation of objective response (either a CR or a PR), as determined by BICR and the investigator according to RANO for patients with primary CNS tumours, RECIST v1.1 for extra cranial tumors, and INRC for neuroblastoma.

As used herein, “clinical benefit rate” (CBR) is defined as the proportion of patients with CR, PR, or SD at 6 months after the first dose of alectinib, as determined by BICR and the investigator according to RANO for patients with primary CNS tumours, RECIST v1.1 for extracranial tumours, and INRC for neuroblastoma.

The term “survival” refers to the patient remaining alive, and includes overall survival as well as progression-free survival.

As used herein, “progression-free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease. In some embodiments, PFS is defined as defined as the time from first dose of alectinib to the first occurrence of disease progression or death from any cause whichever occurs first, as determined by blinded independent central review and the investigator according to RANO for patients with primary CNS tumors, RECIST v1.1 scale for extracranial tumors, and INRC for neuroblastoma.

As used herein, “disease-free survival” (DFS) refers to the time from randomization to the first documented recurrence of disease or new primary disease—as determined by the investigator through use of an integrated assessment of radiographic data, biopsy sample results (if clinically feasible), and clinical status—or death from any cause, whichever occurs first.

As used herein, “overall survival” (OS) refers to defined as the time from the first dose of alectinib to the date of death due to any cause.

By “extending survival” is meant increasing overall or progression-free survival in a treated patient relative to an untreated patient (i.e. relative to a patient not treated with the medicament), and/or relative to a patient treated with an approved anti-tumour agent.

As used herein, “delaying progression” of a disease means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, “preventing” means to reduce the likelihood of disease recurrence as compared to treatment with platinum-based chemotherapy.

The “hazard ratio” is the probability of an event in a treatment group relative to the control group probability over a unit of time. For example, a hazard ratio of 0.16 means that alectinib provides 84% risk reduction in terms of a particular event compared to the control group.

For example, in some embodiments, administration of alectinib increases the subject's likelihood of having an objective response (e.g., a CR), extends the subject's PFS, extends the subjects DFS, extends the subject's OS, and/or extends the subject's DOR as compared to no treatment or treatment without alectinib.

In some embodiments, alectinib increases the subject's likelihood of having an objective response as compared to no treatment or treatment without alectinib. In some embodiments, alectinib increases the subject's likelihood of having a CR as compared to no treatment or treatment without alectinib. In some embodiments, alectinib extends the subject's PFS as compared to no treatment or treatment without alectinib. In some embodiments, alectinib extends the subject's DFS as compared to no treatment or treatment without alectinib. In some embodiments, alectinib extends the subject's OS as compared to no treatment or treatment without alectinib. In some embodiments, alectinib extends the subject's DOR as compared to no treatment or treatment without alectinib. In some embodiments, administration of alectinib to the subject extends the subject's ORR as compared to no treatment or treatment without alectinib.

In some embodiments, benefit from treatment with alectinib includes an increase in OS. In some embodiments, benefit from treatment with alectinib includes an increase in DFS.

In some embodiments of the disclosure, administration of alectinib to the subject extends the subject's DFS as compared to no treatment or treatment without alectinib. For example, administration of alectinib to the subject may extend the subject's DFS by from about 1 month to about 5 months, by from about 2 months to about 4 months, by from about 2.1 months to about 3.9 months, by from about 2.5 months to about 3.5 months, or by from about 2.8 months to about 3.4 months as compared to administration of the platinum-based chemotherapy without alectinib (e.g., by about 1 month, 1.1 months, 1.2 months, 1.3 months, 1.4 months, 1.5 months, 1.6 months, 1.7 months, 1.8 months, 1.9 months, 2 months, 2.1 months, 2.2 months, 2.3 months, 2.4 months, 2.5 months, 2.6 months, 2.7 months, 2.8 months, 2.9 months, 3 months, 3.1 months, 3.2 months, 3.3 months, 3.4 months, 3.5 months, 3.6 months, 3.7 months, 3.8 months, 3.9 months, 4 months, 4.1 months, 4.2 months, 4.3 months, 4.4 months, 4.5 months, 4.6 months, 4.7 months, 4.8 months, 4.9 months, or 5 months).

In some embodiments, administration of alectinib to the subject reduces the subject's likelihood of disease recurrence as compared to no treatment or treatment without alectinib. For example, administration of alectinib to the subject reduces the subject's likelihood of disease recurrence by about 5%, 10%, 15%, 20%, 25%, 30%, 35%, 40%, 45%, 50%, 55%, 60%, 65%, 70%, 75%, 80%, 85%, 90%, 95%, 99%, 100% as compared to no treatment or treatment without alectinib. Preferably, administration of alectinib to the subject reduces the subject's likelihood of disease recurrence by about 60 to 90%, such as 75% to 85% as compared to no treatment or treatment without alectinib.

In some instances, the method further includes administering to the patient an effective amount of a second therapeutic agent. In some instances, the second therapeutic agent is selected from the group comprising chemotherapeutic agents, hormonal therapeutic agents, immunotherapeutic agents, molecular targeting agents, or the like.

Examples of the “chemotherapeutic agents” include an alkylating agent, a platinum formulation, a metabolic antagonist, a topoisomerase inhibitor, an anticancer antibiotic substance, and an anticancer agent derived from plant, etc. Examples of the “alkylating agent” include nitrogen mustard, nitrogen mustard-N-oxide hydrochloride, chlorambucil, cyclophosphamide, ifosfamide, thiotepa, carboquone, improsulfan tosylate, busulfan, nimustin hydrochloride, mitobronitol, melphalan, dacarbazine, ranimustin, estramustin sodium phosphate, triethylene melamine, carmustin, lomustin, streptozocin, pipobroman, etoglucid, altretamin, ambamustin, dibrospidium hydrochloride, fotemustin, prednimustin, pumitepa, ribomustin, temozolomid, treosulfan, trophosphamide, zinostatin stimalamer, carboquone, adozelesin, cystemstin, and bizelecin. Examples of the “platinum formulation” include carboplatin, cisplatin, miboplatin, nedaplatin, and oxaliplatin. Examples of the “metabolic antagonist” include mercaptopurine, 6-mercaptopurine riboside, thioinosine, methotrexate, enocitabin, cytarabin, cytarabin ocfosfate, ancitabin hydrochloride, 5-FU based pharmaceuticals (for example, fluorouracil, tegafur, UFT, doxifluridin, carmofur, galocitabin, and emitefur, etc.), aminopterin, calcium leucovorin, tabloid, butocin, calcium folinate, calcium levofolinate, cladribin, emitefur, fludarabin, gemcitabin, hydrocycarbamide, pentostatin, piritrexim, idoxuridin, mitoguazon, tiazofurin, and ambamustin. Topoisomerase I inhibitor (for example, irinotecan and topotecan, etc.), topoisomerase II inhibitor (for example, sobuzoxan, etc.). Examples of the “anticancer antiobiotic material” include anthracycline-based anticancer agent (doxorubicin hydrochloride, daunorubicin hydrochloride, acrarubicin hydrochloride, pirarubicin hydrochloride, and epirubicin hydrochloride, etc.), actinomycin D, actinomycin C, mitomycin C, chromomycin A3, bleomycin hydrochloride, bleomycin sulfate, peplomycin sulfate, neocarzinostatin, mitramycin, sarcomycin, carzinophyllin, mitotam, zorubicin hydrochloride, mitoxantrone hydrochloride, and idarubicin hydrochloride, etc. Examples of the “anticancer agent derived from a plant” include vincalkaloid anticancer agent (vinblatin sulfate, vincristin sulfate, and vindecin sulfate), taxan anticancer agent (paclitaxel and docetaxel, etc), etoposide, etoposide phosphate, teniposide, and vinorelbin.

Examples of the “hormonal therapeutic agents” include adrenocortical hormone-based pharmaceuticals (for example, dexamethasone, prednisolone, betamethasone, and triamcinolone, etc.). Of these, prednisolone is preferable.

Examples of the “immunotherapeutic agents (BRM)” include picibanil, krestin, sizofiran, lentinan, ubenimex, interferon, interleukin, macrophage colony stimulating factor, granulocyte colony stimulating factor, lymphotoxin, BCG vaccine, Corynebacterium parvum, levamisole, polysaccharide K, and procodazole.

The “molecular targeting agents” include a “pharmaceutical which inhibits the function of a cell proliferation factor and its receptor,” or the like. Examples of the “cell proliferation factor” can be any substance if only it can promote proliferation of a cell, and the included are a peptide having molecular weight of 20,000 or less which exhibits its activity at low concentration via binding to a receptor. Specific examples thereof include (1) EGF (epidermal growth factor) or a substance which has substantially the same activity [e.g., EGF, heregulin (HER2 ligand) etc.], (2) insulin or a substance which has substantially the same activity [e.g., insulin, IGF (insulin-like growth factor)-1, IGF-2, etc.], (3) FGF (fibroblast growth factor) or a substance which has substantially the same activity [e.g., acidic FGF, basic FGF, KGF (keratinocyte growth factor), FGF-10 etc.], (4) VEGF (vascular endothelial growth factor), (5) other cell proliferation factors [e.g., CSF (colony stimulating factor), EPO (erythropoietin), IL-2 (interleukin-2), NGF (nerve growth factor), PDGF (platelet-derived growth factor), TGFβ (transforming growth factor β), HGF (hepatocyte growth factor), etc.], etc.

The “receptor for cell proliferation factor” can be any receptor if only it has an ability of binding to the cell proliferation factor described above. Specific examples thereof include EGF receptor, heregulin receptor (HER2), insulin receptor, IGF receptor, FGF receptor-1 or FGF receptor-2, HGF receptor (c-met), VEGF receptor, and SCF receptor (c-kit). Examples of the “pharmaceuticals which inhibit the activity of cell proliferation factor” include herceptin (HER2 antibody), GLEEVEC (c-kit, abl inhibitor), and Iressa (EGF receptor inhibitor).

Further, a pharmaceutical which inhibits the activity of a plurality of cell proliferation factors even as a single formulation, or a pharmaceutical which blocks cellular signal produced by cell proliferation factor are also included.

In addition to the pharmaceuticals described above, L-asparaginase, aceglaton, procarbazine hydrochloride, protoporphyrin·cobalt complex, mercury hematoporphyrin·sodium, differentiation-promoting agent (e.g. retinoid, vitamin D, etc.), angiogenesis inhibitor, and α-blocker (e.g., tamsulosin hydrochloride, etc.), etc. can be also used.

### Examples

### Clinical Trial Protocol—Study Design

### Description of Study

The study consists of three parts, all enrolling paediatric participants with solid or CNS tumours with known ALK fusion for whom prior treatment has proven to be ineffective or for whom there is no satisfactory treatment available:
Part 1, dose confirmation and safety run-in: to identify the RP2D for paediatric participants, achieving the exposure observed in the adult population
   This portion will consist of a safety run-in with a minimum of 6 participants (up to approximately 18 participants) to assess the safety, tolerability, and pharmacokinetics of alectinib in the paediatric population. The initial paediatric dose will be assessed and refined by modelling and simulation prospectively.
Part 2, initial expansion: to evaluate the safety and preliminary efficacy of alectinib in newly enrolled paediatric participants treated at the paediatric RP2D. A minimum of 10 participants will be enrolled in this part of the study. If 4 or more participants achieve an objective response to alectinib (as described in Part 2, below), additional expansion will be performed.
Part 3, additional expansion: This portion of the study will enroll approximately 14 additional paediatric participants. An integrated efficacy analysis accounting for every participant treated at paediatric RP2D across all cohorts will be used for the confirmation of the efficacy of alectinib.

A minimum of 10 participants with solid tumour and 10 participants with primary CNS tumour treated at the RP2D will be enrolled across the study.

Alectinib will be administered orally or via feeding tube to participants BID on Days 1-28 of each 28-day cycle. All participants will be closely monitored for adverse events (regardless of relationship to study drug) throughout the study and for at least 28 days after the final dose of study treatment or until initiation of a new anti-cancer therapy, whichever occurs first.

Molecular determination of ALK-fusion positivity in tumour tissue acquired at diagnosis or at the time of relapse (regardless of the number of lines of treatment) is required prior to participant enrolment either by local testing or central testing at Foundation Medicine, Inc. Local testing of tumour tissue must be performed using a Sponsor-approved test (e.g., next-generation sequencing [NGS] assay) at a Clinical Laboratory Improvement Amendments (CLIA) or equivalently certified laboratory. For participants enrolled by means of local molecular testing, an archival tumour tissue from diagnosis or, preferably, from relapsed disease (preferably from the same tissue block and unless medically contraindicated) is required to be submitted to the designated study central laboratory for independent retrospective central testing at Foundation Medicine, Inc. or an alternative, approved central assay laboratory for that region. Exceptions for retrospective central testing may be granted at the discretion of the Medical Monitor.

Participants who do not meet the criteria for participation in this study (screen failure) may qualify for one re-screening opportunity (for a total of two screenings per participant) at the investigator's discretion. Participants are not required to re-sign the consent form if they are re-screened within 28 days after previously signing the consent form. The investigator will record reasons for screen failure in the screening log.

An iDMC will be established for safety monitoring at predefined study milestones and approximately every 6 months.

### Part 1: Dose Confirmation and Safety Run-In

### Dose Confirmation

The dose confirmation portion of the study will consist of a safety run-in assessment combined with prospective PK assessment. The starting dose is determined by popPK model to achieve the target exposure in the adults. It is anticipated that a minimum of approximately 6 participants up to approximately 18 participants will be enrolled in Part 1. Participants will receive fixed doses of alectinib based on body weight ranges).

Owing to the limitations of predicting exposure in children who are <2 years old, before enrolment of participants <2 years old, the investigator must discuss a participant's case with the Medical Monitor until at least 3 participants who are ≥2-6 years old have completed one cycle of treatment. The first participant enrolled must complete at least 8 days of treatment without a DLT before subsequent participants can enroll. If a dose adjustment is required because of toxicity, the same approach will be taken for the first participant at the new dose level. Participants will be closely monitored for adverse events during a DLT assessment window of one cycle (28 days). Participants who discontinue from the study prior to completing the DLT assessment window or who complete <80% (i.e., <44 doses) of the prescribed therapy for that cycle for reasons other than a DLT will be considered non-evaluable. DLT non-evaluable participants will be replaced, unless the Medical Monitor determines that replacement is not necessary for a dose adjustment decision. The Medical Monitor will review all participants who take prohibited therapies, foods, or supplements or who receive supportive care during the DLT assessment window that would confound the evaluation of DLTs (not including supportive care described below as part of the DLT definition) to determine DLT evaluability and whether such participants will be replaced.

Participants who experience a DLT during the assessment window (i.e., within the first 28 days) are not permitted to continue study drug at their current dose. The investigator should use guidance provided to determine whether a participant should permanently discontinue study drug or withhold study drug until adequate resolution of the toxicity.

All participants in the safety run-in enrolled at the paediatric RP2D of alectinib, irrespective of diagnosis, will be eligible for the primary efficacy analysis.

Alectinib pharmacokinetics will be monitored prospectively to ensure that the observed exposures of alectinib are within the predicted range. The popPK model (and ultimately PBPK when sufficient data become available) will be continuously updated with the emerging PK data from all enrolled paediatric participants, and the dose will be adjusted if required to ensure exposures reach the target range. Dose level escalation or de-escalation (if required) will be decided following retrospective review of PK and safety data at the end of the DLT assessment window

The paediatric RP2D for each body weight category will be confirmed if the following occur:
The exposure observed at steady state (AUC ss-12 hr) is in compliance with the exposure ranges of 3150-12800 ng·h/mL for alectinib and 1490-5300 ng·h/mL for M4 (its active metabolite) in at least 6 paediatric participants enrolled, including a minimum of 3 participants who are <2 years old. These 3 participants can be counted as part of the total 6 paediatric participants if enrolment allows or can be enrolled at a later time point when RP2D is assessed in participants ≥2 years old. More than 3 participants who are <2 years old may be required depending on the close monitoring of the PK profiles. In case the required total number of participants <2 years of age is not reached, the enrolment of children ≥2 years old in the initial expansion portion of the study may commence on the basis of the established RP2D for children ≥2 years old.
No more than one (>1) DLT is observed in the first 3 participants enrolled treated at the same dose level (≥2 years old), and in the additional 3 participants subsequently enrolled and treated at the same dose level (including a minimum of 3 participants <2 years old). If more than one DLT occurs among the first 6 participants, a revised dose level will be decided in light of available PK data.
If required, following dose adjustment (escalation or de-escalation): No DLT is observed for at least 3 participants treated at the adjusted dose level. Or, if one or more (≥1) DLT is observed in the first 3 participants enrolled, the dose will be further adjusted and no DLT should be observed in the subsequent 3 additional participants treated at the adapted dose level.

During the study, the maximum dose administered will be 600 mg BID (regardless of dose level or participant body weight), corresponding to the adult recommended dose. Following the first cycle, participants will undergo DLT assessment.

It is anticipated that a minimum of 6 participants up to approximately 18 participants will be enrolled in this phase.

### Definition of Dose-Limiting Toxicity

During the safety run-in phase, any one of the following events will be considered a DLT if it occurs during the DLT assessment window and is assessed by the investigator to be related or possibly related to alectinib (rules for attributing causality are defined below in ‘Adverse events’). Adverse events identified as DLTs, as defined below, must be reported to the Sponsor within 24 hours.

The following will be considered as a DLT:
Any treatment-related death
Grade 4 neutropenia (ANC<500 cells/mm³ which is equivalent to ANC<0.5×10⁹/L) lasting at least 7 days
Febrile neutropenia: ANC<1000 cells/mm³ (which is equivalent to ANC<1×10⁹/L) with a single temperature of >38.3° C. (101° F.) or a sustained temperature of ≥38° C. (100.4° F.) for more than 1 hour
Grade 4 anaemia
Grade 4 thrombocytopenia (i.e., platelet count <25.0×10⁹/L) or Grade 3 thrombocytopenia associated with clinically significant bleeding or any thrombocytopenia requiring platelet transfusion.
   Of note, the post-surgical platelet threshold for platelets transfusion could be higher. In that case, the investigator should contact and discuss with the Medical Monitor prior to reporting.
Elevation of serum hepatic transaminase (ALT or AST) of Grade 3 according to NCI CTCAE v5.0 for >72 hours or Grade 4 event lasting for any duration of time not attributable to disease progression
   For participants with Grade ≤2 hepatic transaminase levels at baseline as a result of liver metastases, Grade 3 according to NCI CTCAE v5.0 lasting for >72 hours or Grade 4 event lasting for any duration of time not attributable to disease progression
Grade ≥3 serum bilirubin elevation
Any Grade ≥3 non-hematologic toxicity, with the following exceptions:
   Grade 3 nausea, vomiting, or diarrhoea that responds to SOC therapy in ≤3 days
   Grade 3 fatigue lasting ≤3 days
   Grade 3 laboratory abnormalities that are asymptomatic and are considered by the investigator not to be clinically significant
Delay of >14 days in starting Cycle 2 (beyond Day 42) due to slow recovery from thrombocytopenia (platelet count <75×10⁹/L) or neutrophil count <0.75×10⁹/L
   Delays will be taken into account for subsequent dosing decisions.
Any study drug-related event that results in a dose delay beyond Day 42

Participants who experience a DLT during the assessment window (i.e., in the first 28 days) are not permitted to continue drug at their current dose. The investigator should use guidance provided to determine whether a participant should permanently discontinue study drug or withhold study drug until adequate toxicity resolution. If a participant resumes treatment, the starting dose will be at a reduced dose.

### Part 2: Initial Expansion for Early Efficacy Evaluation

After selection of the alectinib paediatric RP2D, at least 10 additional participants with ALK fusion-positive solid tumours or CNS tumours will be enrolled.

After enrolment of 10 paediatric participants treated at the paediatric RP2D, additional expansion will be allowed if at least 4 of 10 participants experience a confirmed objective response (either a CR or a PR), assessed by BICR.

Response criteria, assessment time points, and primary efficacy endpoints for each disease are listed in Table 1.

TABLE 1
Response Criteria, Assessment Timepoints, and Primary Efficacy Endpoints

| | | | Primary Efficacy |
|---|---|---|---|
| | | | Endpoint for |
| | | | Determination of |
| | | | Subsequent Cohort |
| Disease | Response Criteria | Assessment Timepoint | Expansion |
| CNS tumours: primary | RANO (see below) | Every two cycles for the | Objective response |
| Solid tumours | RECIST v1.1 for | first 12 months and | rate at 6 months |
| | participants with | every three cycles | |
| | other solid tumours | thereafter | |
| | (see below) | | |
| Neuroblastoma | INRC (see below) | | |

CNS = central nervous system; INRC = International Neuroblastoma Response Criteria; RANO = Response Assessment in Neuro-Oncology; RECIST v1.1 = Response Evaluation Criteria in Solid Tumours, Version 1.1.
Note:
Each cycle is 28 days in length.

### Part 3: Additional Expansion Phase

The decision to expand in Part 3 will take into account the safety and tolerability of the regimen in addition to the number of responders, as defined above.

A minimum of 14 additional participants with ALK fusion-positive tumours treated at the paediatric RP2D will be enrolled in Part 3.

After a minimum of 30 paediatric participants treated at RP2D are enrolled, final tumour assessments will be performed utilizing the same response criteria used in Part 2. Participants dosed at the paediatric RP2D treated in Part 1 will be accounted in the final efficacy analysis (see ‘Statistical Considerations and Analysis Plan’ below). A minimum of 10 participants with primary CNS tumours and 10 participants with solid tumours, enrolled at RP2D are required in order to conduct the final efficacy analysis.

### Stopping Rule

A temporary dosing halt and enrolment pause will be applied if any Grade 5 toxicities attributable to study drug occur during the initial dose confirmation and expansion phases of the study. All participants must stop dosing unless evidence of clinical benefit is demonstrated (e.g., decreased tumour-related symptoms, reduced tumour burden, or evidence of stable disease) and has already been observed in the investigator's opinion.

Dosing and enrolment may not resume in the participants until the iDMC recommends resumption of dosing and enrolment after review of the adverse event(s).

### End of Study and Length of Study

The end of this study is defined as the date when the last participant, last visit occurs (LPLV) or 5 years after the last participant is enrolled and treated with study drug, whichever occurs first. For an individual participant, the completion of the study (i.e., the last visit) will occur when the participant withdraws consent, has completed follow-up, has been lost to follow-up, dies, or when the study is stopped.

The primary analysis will be conducted after a minimum of 30 paediatric participants treated at the RP2D across Parts 1, 2 and 3 and a minimum of 10 children with primary CNS tumours and 10 children with solid tumours, treated at the RP2D have been enrolled and followed for at least 6 months.

### Participants

A minimum of 30 children and adolescents with ALK fusion-positive solid or CNS tumours for whom prior treatment has proven to be ineffective (i.e., relapsed or refractory), or for whom there is no satisfactory treatment available will be enrolled in this study.

Participant eligibility will be assessed during a 28-day screening period. Signed informed consent and assent must be obtained prior to enrolment.

### Inclusion Criteria

Participants must meet the following criteria for study entry:
Informed consent and assent
Age at study entry <18 years old
Ability to comply with the study protocol in the investigator's judgment
Histologically confirmed diagnosis of CNS or solid tumours harbouring ALK gene fusions as determined locally by an appropriately validated assay performed in a CLIA-certified or equivalently-accredited diagnostic laboratory, or centrally by a Foundation Medicine Clinical Trial Assay (CTA) or the alternative, approved central laboratory for that region.
   For participants enrolled via local molecular testing, the initial assay report should be submitted to the sponsor for inclusion in the central repository.
   For the enrolment of tumour subtypes, a minimum number of 10 participants with solid tumour and 10 participants with CNS tumour is required.
Disease status: prior treatment proven to be ineffective (i.e., relapsed or refractory), or for whom there is no satisfactory treatment available. Disease should be measurable and evaluable as defined by RECIST v1.1 for solid tumours, or RANO±bone marrow criteria for primary CNS tumours or INRC criteria (as appropriate, see below).
Available tumour tissue for submission to the Sponsor from active disease, obtained subsequent to last anti-cancer therapy regimen administered and obtained prior to study enrolment, or willingness to undergo a core or excisional biopsy sample collection prior to enrolment
   Participants must submit a tissue block or 5-10 slides containing unstained, freshly cut, serial sections available for submission (preferably from the same tissue block as used for local testing if ALK status was determined locally).
   Fine-needle aspirations, brush biopsies, bone metastasis samples, and lavage samples are not acceptable.
   Participants with <5 slides available, or whose tumour tissue does not otherwise meet criteria above, may be eligible for study entry at the discretion of the investigator following advice from the Medical Monitor.
Life expectancy ≥8 weeks, in the investigator's judgment
Adequate performance status:
   Participants <16 years old: Lansky Performance Status ≥50%
   Participants ≥16 years old: Karnofsky Performance Status ≥50%
   Note: Neurologic deficits in participants with CNS tumours must be relatively stable for at least 7 days prior to study enrolment. If a deficit is due to tumour or is a post-surgical adverse event that is deemed by the investigator to be temporary, a participant with a performance status below 50% may be eligible at the discretion of the Investigator following advice from the Medical Monitor.
Adequate end-organ function defined by the following laboratory results obtained within 28 days prior to initiation of study drug
Adequate bone marrow function, as defined by the following laboratory parameters obtained within 28 days prior to initiation of study drug:
   Hemoglobin ≥8 g/dL (transfusion allowed)
   Peripheral ANC≥1.00×10⁹/L and no granulocyte colony-stimulating factor (G-CSF) support for at least a duration of >5 half-lives of the G-CSF product from last injection
   Platelet count ≥75×10⁹/L (unsupported for 72 hours)
   If bone marrow metastatic disease:
      ANC≥0.75×10⁹/L.
      Platelets: not known to be refractory to platelets transfusions
Willingness and ability (participant and/or caregiver) to complete clinical outcome assessments throughout the study using either electronic, paper, or interviewer methods
For females of childbearing potential: agreement to remain abstinent (refrain from heterosexual intercourse) or use contraception, and agreement to refrain from donating eggs, as defined below:
   Females must remain abstinent or use two adequate methods of contraception, including at least one method with a failure rate of <1% per year prior to study entry, during the treatment period and for at least 3 months after the final dose of study drug: Females must refrain from donating eggs during this same period.
   A female is considered to be of childbearing potential if she is postmenarchal, has not reached a postmenopausal state (>12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis). The definition of childbearing potential may be adapted for alignment with local guidelines or regulations.
   Females of childbearing potential must have a negative serum pregnancy test during screening and be neither breastfeeding nor intending to become pregnant during study participation.
   Examples of contraceptive methods with a failure rate of <1% per year include bilateral tubal ligation, male sterilization, established proper use of hormonal contraceptives that inhibit ovulation, hormone-releasing intrauterine devices, and copper intrauterine devices.
   A barrier method may be used as the second contraceptive method with the use of spermicide.
   The reliability of sexual abstinence should be evaluated in relation to the duration of the clinical trial and the preferred and usual lifestyle of the participant. Periodic abstinence (e.g., calendar, ovulation, symptothermal, or postovulation methods) and withdrawal are not adequate methods of contraception. If required per local guidelines or regulations, locally recognized adequate methods of contraception and information about the reliability of abstinence will be described in the local Informed Consent Form.
For males who are not surgically sterile: agreement to remain abstinent (refrain from heterosexual intercourse) or use contraception, and agreement to refrain from donating sperm, as defined below:
   With a female partner of childbearing potential who is not pregnant, males who are not surgically sterile must remain abstinent or use a condom plus an additional contraceptive method that together result in a failure rate of 1% per year during the treatment period and for at least 3 months after the final dose of alectinib. Males must refrain from donating sperm during this same period.
   With a pregnant female partner, male must remain abstinent or use a condom during the treatment period and for at least 3 months after the final dose of alectinib to avoid exposing the embryo.
   The reliability of sexual abstinence should be evaluated in relation to the duration of the clinical trial and the preferred and usual lifestyle of the participant. Periodic abstinence (e.g., calendar, ovulation, symptothermal, or postovulation methods) and withdrawal are not adequate methods of contraception. If required per local guidelines or regulations, locally recognized adequate methods of contraception and information about the reliability of abstinence will be described in the local Informed Consent Form.

### Exclusion Criteria

Participants who meet any of the following criteria will be excluded from study entry:
Medical history of:
   Prior use of ALK inhibitors
   Any GI disorder that may affect absorption of oral medications, such as mal-absorption syndrome or status post-major bowel resection
   History of organ transplant
   Stem cell infusions (with or without traumatic brain injury) with following:
      Allogeneic (non-autologous) bone marrow or stem cell transplant, or any stem cell infusion including donor leukocyte infusion or boost infusion: <84 days after infusion and/or on chronic immunosuppressive drug.
      Autologous stem cell infusion including boost infusion: <42 days
   History of hypersensitivity to any of the additives in the alectinib drug formulation
   Substance abuse within 12 months prior to screening, in the investigator's judgment
   Familial or personal history of congenital bone disorders, bone metabolism alterations or osteopenia
   Treatment with investigational therapy 28 days prior to initiation of study drug
Current disease status:
   Liver disease as evidenced by the following:
      ALT or AST>3×ULN (≥5×ULN for participants with concurrent liver metastasis)
      Bilirubin ≥1.5×ULN for age (or ≥2.5×ULN if liver infiltrated or metastases)
   Creatinine ≥1.5 ULN for age or an estimated glomerular filtration rate (GFR)<60 mL/min/1.73 m² GFR based on the Schwartz equation (Mian and Schwartz 2017) or as per institutional guidelines
   NCI CTCAE v5.0 Grade ≥3 toxicities attributed to any prior therapy such as radiotherapy (excluding alopecia), which have not shown improvement and are strictly considered to interfere with alectinib
   Co-administration of anti-cancer therapies other than those administered in this study
   Active hepatitis B or C virus (HBV, HBC) or known HIV positivity or AIDS-related illness
   Any clinically significant concomitant disease or condition that could interfere with, or for which the treatment might interfere with, the conduct of the study or the absorption of oral medications or that would, in the opinion of the Principal Investigator, pose an unacceptable risk to the participant in this study
   Any psychological, familial, sociological, or geographical condition potentially hampering compliance with the study protocol requirements and/or follow-up procedures; such conditions should be discussed with the participant before trial entry
   Planned procedure or surgery during the study except as permitted treatment per ‘Permitted therapy’ below
   Infection considered by the investigator to be clinically uncontrolled or of unacceptable risk to the participant upon induction of neutropenia, including participants who are, or should be, on antimicrobial agents for the treatment of active infection such as the following:
      Fungal infection, other than mucosal candidiasis, with <2 weeks of appropriate systemic antifungal therapy.
      Bacterial infection with positive cultures within 7 days prior to dosing.
      Participants who have received <5 days of appropriate therapeutic antibiotic therapy for an identified infection.
      Neutropenic fever considered infection-related within 72 hours prior to dosing
   Pregnant or breastfeeding, or intending to become pregnant during the study or within 3 months after the final dose of alectinib

### Investigational Medicinal Products

The investigational medicinal product (IMP) for this study is alectinib.

The alectinib drug substance used for the manufacture of the drug product is milled hydrochloride salt (designated as RO5424802-002) existing as a crystalline form (Form I).
Systematic name: 9-Ethyl-6,6-dimethyl-8-[4-(morpholin-4-yl) piperidin-1-yl]-11-oxo-6,11-dihydro-5Hbenzo[b]carbazole-3-carbonitrile hydrochloride
Molecular Formula: C₃₀H₃₄N₄O₂ HCl
Molecular Weight: 519.08 g/mol (HCl salt); 482.62 g/mol (free base)
Appearance: fine powder or powder with lumps
Colour: white to yellow reddish white
Physical state: solid

Alectinib capsules should be administered orally BID, with body weight adjusted/adapted dosage, with food in the morning and evening.

For children unable to swallow intact capsules, ad-hoc preparation of a suspension using the available hard capsule is possible.

### Study Treatment Dosage, Administration, and Compliance

Alectinib is available in 20-mg and 150-mg hard capsules and should be administered orally BID with food in the morning and evening.

For younger children who have difficulty swallowing intact capsules, an alternative method of preparation and administration of an oral suspension (using the available 150 mg capsules) is available.

The starting dose in Part 1 for participants weighing ≥10 kg is listed in Table 2 (capsules) and Table 3 (suspension), and for participants weighing between 3.5 and 10 kg in Table 4 (capsules) and Table 5 (suspension). For participants less than 3.5 kg, discussion with the Medical Monitor on a case by case basis is warranted. Alectinib can be administered orally or by means of a feeding tube as a suspension.

If a participant misses a dose, it can be taken within 6 hours of the scheduled time. If the time is greater than 6 hours, or if the participant vomits all or part of the dose, the participant should wait until the next scheduled time and take the next scheduled dose. Participants should not take two doses at the same time to make up for a missed dose.

Participants will receive a daily dose of alectinib by range of body weight and the following adjustment may be done.

TABLE 2
Starting Dose Recommendation with Capsules for
Participants with Body Weight ≥ 10 kg for Part 1

| | | Starting | First Dose | Second Dose |
|---|---|---|---|---|
| | Corresponding | Dose | Reduction | Reduction |
| BW Category | age^{a} (year) | BID | (−25%) | (−33%) |
| ≥10−<15 kg | 1-3.5 | 190 mg | 140 mg | 100 mg |
| ≥15−<20 kg | 3.5-5.8 | 230 mg | 170 mg | 120 mg |
| ≥20−<25 kg | 5.8-7.8 | 270 mg | 210 mg | 140 mg |
| ≥25−<30 kg | 7.8-9.4 | 300 mg | 230 mg | 150 mg |
| ≥30−<35 kg | 9.4-10.7 | 340 mg | 250 mg | 170 mg |
| ≥35−<40 kg | 10.7-11.8 | 380 mg | 290 mg | 190 mg |
| ≥40−<45 kg | 11.8-12.8 | 600 mg | 450 mg | 300 mg |
| ≥45−<50 kg | 12.8-14 | 600 mg | 450 mg | 300 mg |
| ≥50−<55 kg | 14-15.8 | 600 mg | 450 mg | 300 mg |
| ≥55−<60 kg | >15.8 | 600 mg | 450 mg | 300 mg |

BID = twice daily; BW = body weight; CDC = Centers for Disease Control and Prevention.
Note:
Suspension will be provided to participant unable to swallow whole capsules. For detailed information please refer to the Pharmacy Manual and Instruction for Use.
^{a}Extrapolated from healthy male and female weight-age medians (CDC growth charts).

TABLE 3
Starting Dose Recommendation with Suspension for
Participants with Body Weight ≥10 kg for Part 1

| | | Starting | First Dose | | Second Dose | |
|---|---|---|---|---|---|---|
| | Corresponding | Dose | Reduction | | Reduction | |
| BW Category | age^{a} (year) | BID | (−25%) | | (−33%) | |
| ≥10−<15 kg | 1-3.5 | 114 mg | 85 | mg | 57 | mg |
| ≥15−<20 kg | 3.5-5.8 | 138 mg | 103 | mg | 69 | mg |
| ≥20−<25 kg | 5.8-7.8 | 162 mg | 121 | mg | 81 | mg |
| ≥25−<30 kg | 7.8-9.4 | 180 mg | 135 | mg | 90 | mg |
| ≥30−<35 kg | 9.4-10.7 | 204 mg | 153 | mg | 102 | mg |
| ≥35−<40 kg | 10.7-11.8 | 228 mg | 171 | mg | 114 | mg |
| ≥40−<45 kg | 11.8-12.8 | 359 mg | 269 | mg | 180 | mg |
| ≥45−<50 kg | 12.8-14 | 359 mg | 269 | mg | 180 | mg |
| ≥50−<55 kg | 14-15.8 | 359 mg | 269 | mg | 180 | mg |
| ≥55−<60 kg | >15.8 | 359 mg | 269 | mg | 180 | mg |

BID = twice daily; BW = body weight; CDC = Centers for Disease Control and Prevention.
Note:
Suspension will be provided to participant unable to swallow whole capsules. For detailed information please refer to the Pharmacy Manual and Instruction for Use.
^{a}Extrapolated from healthy male and female weight-age medians (CDC growth charts).

TABLE 4
Starting Dose Recommendation with Capsules for Participants
with Body Weight between 3.5 and 10 kg for Part 1

| | | | | First Dose | | Second Dose | |
|---|---|---|---|---|---|---|---|
| | Corresponding | Dose | | Reduction | | Reduction | |
| BW Category | age^{a} (months) | BID | | (−25%) | | (−33%) | |
| ≥3.5−<5 kg | 0-2 | 80 | mg | 60 | mg | 40 | mg |
| ≥5−<7.5 kg | 2-6 | 100 | mg | 75 | mg | 50 | mg |
| ≥7.5−<10 kg | 6-12 | 140 | mg | 105 | mg | 70 | mg |

BID = twice daily; BW = body weight; CDC = Centers for Disease Control and Prevention.
Note:
Suspension will be provided to participant unable to swallow whole capsules. For detailed information please refer Pharmacy Manual and Instruction for Use.
^{a}Extrapolated from healthy male and female weight-age medians (CDC growth charts)

TABLE 5
Starting Dose Recommendation with Suspension for Participants
with Body Weight between 3.5 and 10 kg for Part 1

| | | | First Dose | Second Dose |
|---|---|---|---|---|
| | Corresponding | Dose | Reduction | Reduction |
| BW Category | age^{a} (months) | BID | (−25%) | (−33%) |
| ≥3.5−<5 kg | 0-2 | 48 mg | 36 mg | 24 mg |
| ≥5−<7.5 kg | 2-6 | 60 mg | 45 mg | 30 mg |
| ≥7.5−<10 kg | 6-12 | 84 mg | 63 mg | 42 mg |

BID = twice daily; BW = body weight; CDC = Centers for Disease Control and Prevention.
Note:
Suspension will be provided to participant unable to swallow whole capsules. For detailed information please refer Pharmacy Manual and Instruction for Use.
^{a}Extrapolated from healthy male and female weight-age medians (CDC growth charts)

### Permitted Therapy

Participants are permitted to use the following therapies during the study:
Oral contraceptives with a failure rate of <1% per year
Anticoagulants and antithrombotic agents (i.e., Coumadin-derived anticoagulants, unfractionated heparin or low-molecular-weight heparins, and paediatric doses of aspirin as per local recommendations)
Acetaminophen (adjusted to paediatric participants as per local recommendations)
Gastric pH elevating medications (such as proton pump inhibitors, H2 blockers, or antacids)
Hormone-replacement therapy
Local therapy (e.g., surgery, stereotactic radiosurgery, radiofrequency ablation) as outlined below:
   Participants experiencing a mixed response requiring local therapy for control of three or fewer lesions may still be eligible to continue study treatment at the investigator's discretion following advice from the Medical Monitor. Participants who receive local therapy directed at a target lesion will no longer be evaluable for radiographic response but will remain evaluable for progression.
   Alectinib has no inhibitory activity on FGFR2; however, there is a known negative chronotropic effect of alectinib that may result in bradycardia. This may be of relevance for major elective surgeries involving general anaesthesia, and the anesthesiologist should be informed of this. Based on PK considerations, alectinib treatment should be stopped approximately 5 days prior to the planned surgery involving general anesthesia. Postoperatively, the decision to reinitiate treatment with alectinib should be based on clinical assessment of a participant's satisfactory recovery from surgery. For participants who need general anesthesia for tumour assessments or other radiological interventions, it is not necessary to hold alectinib; however, the anesthesiologist should be informed of the risk of bradycardia caused by the drug.

Premedication with antihistamines, antipyretics, and/or analgesics may be administered at the discretion of the investigator. In general, investigators should manage a participant's care with supportive therapies as clinically indicated, per local standard practice. Participants who experience study drug-associated symptoms may be treated symptomatically with acetaminophen, ibuprofen, diphenhydramine, and/or H2-receptor antagonists (e.g., famotidine, cimetidine), or equivalent medications per local standard practice. Serious study drug-associated events manifested by dyspnea, hypotension, wheezing, bronchospasm, tachycardia, reduced oxygen saturation, or respiratory distress should be managed with supportive therapies as clinically indicated (e.g., supplemental oxygen and β₂-adrenergic agonists).

### Prohibited Therapy

Use of the following therapies (e.g., prescription drugs, over-the-counter drugs, herbal or homeopathic remedies, nutritional supplements) is prohibited during the study and for at least 14 days prior to the first dose of alectinib, unless otherwise specified below.

Exceptions to restrictions of the concomitant therapies listed below (e.g., prescription drugs, over-the-counter drugs, herbal or homeopathic remedies, nutritional supplements) may be made if the rationale is discussed and documented between the investigator and the Sponsor's clinical pharmacologist.
Systemic immunosuppressive drugs, cytotoxic or chemotherapeutic agents (other than study drug treatment), ergot derivatives, probenecid, and bile acid-binding resins during study treatment
Systemic chemotherapy
Radiotherapy and radionuclide therapy except for palliative radiotherapy to bone lesions or for pain control as described below:
   If palliative radiotherapy is indicated for bone metastases, palliative radiotherapy may start within 24 hours of the last dose of alectinib, unless, in the judgment of the investigator, participant safety will require a longer washout period prior to palliative radiotherapy. Dosing of alectinib may resume with the resolution of any radiation toxicity to ≤Grade 1 or better.
Additional investigational drug (except during the follow-up period)
Strong CYP3A inhibitors/inducers in Part 1, Cycle 1 (all participants) and Part 1, Cycle 2 (if received dose adjustment in the beginning of Cycle 2) only

### Study Assessments

Participants will be closely monitored for safety and tolerability throughout the study.

Participants should be assessed for toxicity at each study visit; dosing will occur only if the clinical assessment and local laboratory test values are acceptable.

### Medical History, Baseline Conditions, Concomitant Medication, and Demographic Data

Medical history, including clinically significant diseases, surgeries, dental history, cancer history (including prior cancer therapies and procedures), menstrual history, fertility history, and puberty history, smoking history, and use of alcohol and drugs of abuse, will be recorded at baseline. In addition, all medications (e.g., prescription drugs, over-the-counter drugs, vaccines, herbal or homeopathic remedies, nutritional supplements) used by the participant within 14 days prior to initiation and until 30 days after the final dose of study treatment will be recorded. At the time of each follow-up physical examination, an interval medical history should be obtained and any changes in medications and allergies should be recorded.

Demographic data will include age, sex, and self-reported race/ethnicity (only where permitted by local regulations).

A complete physical examination, performed at screening and other specified visits, should include an evaluation of the head, eyes, ears, nose, throat, and teeth, and the cardiovascular, dermatologic, musculoskeletal, respiratory, GI, genitourinary, and neurologic systems. Any abnormality identified at baseline should be recorded on the General Medical History and Baseline Conditions eCRF.

During the treatment period, weight will be measured at the beginning of each cycle. During the follow-up period, weight should be measured every 3 months.

During the treatment period, height, head circumference (until the age of 3 years), and Tanner stage should be measured every three cycles (approximately every 3 months) for the first 2 years and then every six cycles (approximately 6 months). Tanner staging should be performed until the participant has reached Tanner Stage V or the end of the study.

During the follow-up period, height, head circumference, and Tanner stage should be measured every 3 months.

In order to monitor for potential visual disturbances during treatment with alectinib age-specific visual acuity testing (which should be reported in Snellen units), or age-appropriate equivalent should be used to evaluate visual acuity as well as colour vision during screening, Day 1 of Cycle 2, and Day 1 of every subsequent cycle, at the treatment discontinuation visit, and as clinically indicated.

A complete physical examination, performed at screening, at the study discontinuation visit and other specified visits should include an evaluation of the head, eyes, ears, nose, and throat, and the cardiovascular, dermatologic, musculoskeletal, respiratory, gastrointestinal, and neurologic systems; genitourinary examinations may be performed if clinically indicated.

A complete neurologic examination should be completed Day 1 of each cycle, including at baseline. Any abnormality identified at baseline should be recorded on the General Medical History and Baseline Conditions eCRF.

For participants without a full set of permanent teeth, an oral exam and oral medical history should be conducted during screening, at Cycle 7, Cycle 13 then yearly and at study discontinuation visit.

Age-appropriate equipment should be used to obtain vital signs and assessments and adjusted for growth. Vital signs will include measurements of respiratory rate, pulse rate, systolic and diastolic blood pressures, oxygen saturation by pulse oximetry, and temperature.

### Tumour and Response Evaluations

All known sites of disease must be documented at screening and re-assessed at each subsequent tumour evaluation. Baseline tumour assessments should be performed within 28 days prior to initiation of study drug (Day 1 of Cycle 1); subsequent tumour assessments should be performed in accordance with the schedule of activities. At the investigator's discretion, unscheduled tumour assessments may be performed at any time if progressive disease is suspected.

Response will be assessed by the investigator on the basis of physical examinations, computed tomography (CT) scans, and other modalities where relevant (e.g., magnetic resonance imaging [MRI] scans, brain scans, bone marrow examinations, fluorodeoxyglucose-positron emission tomography (FDG-PET), metaiodobenzoguanidine [MIBG] scan), according to RANO, INRC, and RECIST v1.1 (see below). Tumour assessment imaging should include all areas of known disease.

An objective response should be confirmed by repeat assessments ≥4 weeks after initial documentation. The same radiographic procedure used to assess disease sites at screening should be used throughout the study (e.g., the same contrast protocol for CT scans). To the extent that is feasible, assessments should be performed by the same evaluator to ensure internal consistency across visits.

Bone marrow examinations should include biopsy and aspirate for morphology (flow studies are optional) and are required at screening for staging purposes unless a bone marrow examination has been performed within 3 months prior to Day 1. If positive at screening, a subsequent bone marrow examination is required only to confirm a CR.

Participants with other solid tumours who have a clinical suspicion of bone marrow involvement should also have bilateral bone marrow aspirations (BMAs) and/or bone marrow biopsies performed at screening. Participants who have documented bone marrow involvement at screening should have subsequent bone marrow evaluations performed at each response assessment until the first negative bone marrow evaluation.

Extracranial tumours will be evaluated for disease response and progressive disease with RECIST v1.1 (see below)

Neuroblastoma will be evaluated for disease response and progressive disease with INRC (see below).

Primary CNS tumours will be evaluated for disease response and progressive disease with RANO (see below).

Participants who enroll with evaluable but not measurable disease, will undergo a modified response assessment. For such participants, partial response cannot be determined; therefore, response will be limited to CR, SD, or PD.

### 1. RECIST v1.1

Selected sections from the Response Evaluation Criteria in Solid Tumors (RECIST), Version 1.1 (Eisenhauer et al., 2009, Eur J Cancer, 45:228-47) are presented below, with slight modifications.

At baseline, tumour lesions/lymph nodes will be categorized as measurable or nonmeasurable—as described below. All measurable and non-measurable lesions should be assessed at screening and at subsequent protocol-specified tumour assessment timepoints. Additional assessments may be performed as clinically indicated for suspicion of progression.
Measurable tumour legions:
   Tumour Lesions. Tumour lesions must be accurately measured in at least one dimension (longest diameter in the plane of measurement is to be recorded) with a minimum size as follows:
      10 mm by computed tomography (CT) or magnetic resonance imaging (MRI) scan (CT/MRI scan slice thickness/interval no greater than 5 mm).
      10-mm caliper measurement by clinical examination (lesions that cannot be accurately measured with calipers should be recorded as non-measurable)
      20 mm by chest X-ray
   Malignant Lymph Nodes. To be considered pathologically enlarged and measurable, a lymph node must be >15 mm in the short axis when assessed by CT scan (CT scan slice thickness recommended to be no greater than 5 mm). At baseline and follow-up, only the short axis will be measured and followed. See also notes below on “Baseline Documentation of Target and Non-Target Lesions” for information on lymph node measurement.
Non-measurable tumour legions: Non-measurable tumour lesions encompass small lesions (longest diameter <10 mm or pathological lymph nodes with short axis ≥10 but <15 mm) as well as truly nonmeasurable lesions. Lesions considered truly non-measurable include leptomeningeal disease, ascites, pleural or pericardial effusion, inflammatory breast disease, lymphangitic involvement of skin or lung, peritoneal spread, and abdominal mass/abdominal organomegaly-identified by physical examination that is not measurable by reproducible imaging techniques.
Special Considerations Regarding Lesion Measurability: Bone lesions, cystic lesions, and lesions previously treated with local therapy require particular comment, as outlined below:
   Bone Lesions:
      Bone scan, positron emission tomography (PET) scan, or plain films are not considered adequate imaging techniques for measuring bone lesions. However, these techniques can be used to confirm the presence or disappearance of bone lesions.
      Lytic bone lesions or mixed lytic-blastic lesions, with identifiable soft tissue components, that can be evaluated by cross-sectional imaging techniques such as CT or MRI can be considered as measurable lesions if the soft tissue component meets the definition of measurability described above.
      Blastic bone lesions are non-measurable.
   Cystic Lesions: Lesions that meet the criteria for radiographically defined simple cysts should not be considered as malignant lesions (neither measurable nor non-measurable) since they are, by definition, simple cysts. Cystic lesions thought to represent cystic metastases can be considered as measurable lesions, if they meet the definition of measurability described above. However, if non-cystic lesions are present in the same participant, these are preferred for selection as target lesions.
   Lesions with Prior Local Treatment: Tumor lesions situated in a previously irradiated area, or in an area subjected to other loco-regional therapy, are usually not considered measurable unless there has been demonstrated progression in the lesion. Study protocols should detail the conditions under which such lesions would be considered measurable.

All measurements should be recorded in metric notation, using calipers if clinically assessed. All baseline evaluations should be performed as close as possible to the treatment start and never more than 4 weeks before the beginning of the treatment.

The same method of assessment and the same technique should be used to characterize each identified and reported lesion at baseline and during the study. Imaging-based evaluation should always be the preferred option.
Clinical Lesions. Clinical lesions will only be considered measurable when they are superficial and ≥10 mm in diameter as assessed using calipers (e.g., skin nodules). For the case of skin lesions, documentation by colour photography including a ruler to estimate the size of the lesion is suggested.
Chest X-Ray. Chest CT is preferred over chest X-ray, particularly when progression is an important endpoint, since CT is more sensitive than X-ray, particularly in identifying new lesions. However, lesions on chest X-ray may be considered measurable if they are clearly defined and surrounded by aerated lung.
CT, MRI. CT is the best currently available and reproducible method to measure lesions selected for response assessment. This guideline has defined measurability of lesions on CT scan based on the assumption that CT slice thickness is 5 mm or less. When CT scans have slice thickness greater than 5 mm, the minimum size for a measurable lesion should be twice the slice thickness. MRI is also acceptable.
If prior to enrolment it is known that a participant is unable to undergo CT scans with intravenous (IV) contrast because of allergy or renal insufficiency, the decision as to whether a non-contrast CT or MRI (without IV contrast) will be used to evaluate the participant at baseline and during the study should be guided by the tumor type under investigation and the anatomic location of the disease. For participants who develop contraindications to contrast after baseline contrast CT is done, the decision as to whether non-contrast CT or MRI (enhanced or non-enhanced) will be performed should also be based on the tumour type and the anatomic location of the disease, and should be optimized to allow for comparison with the prior studies if possible. Each case should be discussed with the radiologist to determine if substitution of these other approaches is possible and, if not, the participant should be considered not evaluable from that point forward. Care must be taken in measurement of target lesions on a different modality and interpretation of non-target disease or new lesions, since the same lesion may appear to have a different size using a new modality.
Ultrasound. Ultrasound is not useful in assessment of lesion size and should not be used as a method of measurement.
Endoscopy, Laparoscopy, Tumor Markers, Cytology, Histology. The utilization of these techniques for objective tumour evaluation cannot generally be advised.

To assess objective response or future progression, it is necessary to estimate the overall tumour burden at baseline and use this as a comparator for subsequent measurements. Measurable disease is defined by the presence of at least one measurable lesion, as detailed above.

When more than one measurable lesion is present at baseline, all lesions up to a maximum of five lesions total (and a maximum of two lesions per organ) representative of all involved organs should be identified as target lesions and will be recorded and measured at baseline. This means that, for instances in which participants have only one or two organ sites involved, a maximum of two lesions (one site) and four lesions (two sites), respectively, will be recorded. Other lesions (albeit measurable) in those organs will be recorded as non-measurable lesions (even if the size is >10 mm by CT scan).

Target lesions should be selected on the basis of their size (lesions with the longest diameter) and be representative of all involved organs, but in addition should lend themselves to reproducible repeated measurements. It may be the case that, on occasion, the largest lesion does not lend itself to reproducible measurement, in which circumstance the next largest lesion that can be measured reproducibly should be selected.

Lymph nodes merit special mention since they are normal anatomical structures that may be visible by imaging even if not involved by tumour. As noted above, pathological nodes that are defined as measurable and may be identified as target lesions must meet the criterion of a short axis of ≥15 mm by CT scan. Only the short axis of these nodes will contribute to the baseline sum. The short axis of the node is the diameter normally used by radiologists to judge if a node is involved by solid tumor. Nodal size is normally reported as two dimensions in the plane in which the image is obtained (for CT this is almost always the axial plane; for MRI the plane of acquisition may be axial, sagittal, or coronal). The smaller of these measures is the short axis. For example, an abdominal node that is reported as being 20 mm×30 mm has a short axis of 20 mm and qualifies as a malignant, measurable node. In this example, 20 mm should be recorded as the node measurement. All other pathological nodes (those with short axis ≥10 mm but <15 mm) should be considered non-target lesions. Nodes that have a short axis of <10 mm are considered non-pathological and should not be recorded or followed.

A sum of the diameters (longest for non-nodal lesions, short axis for nodal lesions) for all target lesions will be calculated and reported as the baseline sum of diameters. If lymph nodes are to be included in the sum, then, as noted above, only the short axis is added into the sum. The baseline sum of diameters will be used as a reference to further characterize any objective tumour regression in the measurable dimension of the disease.

All other lesions (or sites of disease), including pathological lymph nodes, should be identified as non-target lesions and should also be recorded at baseline. Measurements are not required and these lesions should be followed as “present,” “absent,” or in rare cases “unequivocal progression.”

In addition, it is possible to record multiple non-target lesions involving the same organ as a single item on the Case Report Form (CRF) (e.g., “multiple enlarged pelvic lymph nodes” or “multiple liver metastases”).

### Evaluation of Target Lesions:

This section provides the definitions of the criteria used to determine objective tumor response for target lesions.
CR: Disappearance of all target lesions
   Any pathological lymph nodes (whether target or non-target) must have reduction in short axis to <10 mm.
Partial response (PR): At least a 30% decrease in the sum of diameters of target lesions, taking as reference the baseline sum of diameters
Progressive disease (PD): At least a 20% increase in the sum of diameters of target lesions, taking as reference the smallest sum on study (nadir), including baseline
   In addition to the relative increase of 20%, the sum must also demonstrate an absolute increase of at least 5 mm.
   The appearance of one or more new lesions is also considered progression.
Stable disease (SD): Neither sufficient shrinkage to qualify for PR nor sufficient increase to qualify for PD, taking as reference the smallest sum on study

Lymph Nodes. Lymph nodes identified as target lesions should always have the actual short axis measurement recorded (measured in the same anatomical plane as the baseline examination), even if the nodes regress to <10 mm on study. This means that when lymph nodes are included as target lesions, the sum of lesions may not be zero even if CR criteria are met, since a normal lymph node is defined as having a short axis of <10 mm.

Target Lesions That Become Too Small to Measure. During the study, all lesions (nodal and non-nodal) recorded at baseline should have their actual measurements recorded at each subsequent evaluation, even when very small (e.g., 2 mm). However, sometimes lesions or lymph nodes that are recorded as target lesions at baseline become so faint on CT scan that the radiologist may not feel comfortable assigning an exact measure and may report them as being too small to measure. When this occurs, it is important that a value be recorded on the CRF, as follows:
If it is the opinion of the radiologist that the lesion has likely disappeared, the measurement should be recorded as 0 mm.
If the lesion is believed to be present and is faintly seen but too small to measure, a default value of 5 mm should be assigned and below measurable limit (BML) should be ticked. (Note: It is less likely that this rule will be used for lymph nodes since they usually have a definable size when normal and are frequently surrounded by fat such as in the retroperitoneum; however, if a lymph node is believed to be present and is faintly seen but too small to measure, a default value of 5 mm should be assigned in this circumstance as well and BML should also be ticked).

To reiterate, however, if the radiologist is able to provide an actual measure, that should be recorded, even if it is below 5 mm, and in that case BML should not be ticked.

Lesions That Split or Coalesce on Treatment. When non-nodal lesions fragment, the longest diameters of the fragmented portions should be added together to calculate the target lesion sum. Similarly, as lesions coalesce, a plane between them may be maintained that would aid in obtaining maximal diameter measurements of each individual lesion. If the lesions have truly coalesced such that they are no longer separable, the vector of the longest diameter in this instance should be the maximum longest diameter for the coalesced lesion.

### Evaluation of Non-Target Lesions:

This section provides the definitions of the criteria used to determine the tumor response for the group of non-target lesions. While some non-target lesions may actually be measurable, they need not be measured and instead should be assessed only qualitatively at the timepoints specified in the protocol.
CR: Disappearance of all non-target lesions and (if applicable) normalization of tumor marker level.
   All lymph nodes must be non-pathological in size (<10 mm short axis).
Non-CR/Non-PD: Persistence of one or more non-target lesions and/or (if applicable) maintenance of tumor marker level above the normal limits
PD: Unequivocal progression of existing non-target lesions
   The appearance of one or more new lesions is also considered progression.

When the Participant Also Has Measurable Disease. In this setting, to achieve unequivocal progression on the basis of the non-target disease, there must be an overall level of substantial worsening in non-target disease in a magnitude that, even in the presence of SD or PR in target disease, the overall tumor burden has increased sufficiently to merit discontinuation of therapy. A modest increase in the size of one or more non-target lesions is usually not sufficient to qualify for unequivocal progression status. The designation of overall progression solely on the basis of change in non-target disease in the face of SD or PR of target disease will therefore be extremely rare.

When the Participant Has Only Non-Measurable Disease. This circumstance arises in some Phase III trials when it is not a criterion of study entry to have measurable disease. The same general concepts apply here as noted above; however, in this instance there is no measurable disease assessment to factor into the interpretation of an increase in non-measurable disease burden. Because worsening in non-target disease cannot be easily quantified (by definition: if all lesions are truly non-measurable), a useful test that can be applied when assessing participants for unequivocal progression is to consider if the increase in overall disease burden based on the change in non-measurable disease is comparable in magnitude to the increase that would be required to declare PD for measurable disease, that is, an increase in tumor burden representing an additional 73% increase in volume (which is equivalent to a 20% increase in diameter in a measurable lesion). Examples include an increase in a pleural effusion from “trace” to “large” or an increase in lymphangitic disease from localized to widespread, or may be described in protocols as “sufficient to require a change in therapy.” If unequivocal progression is seen, the participant should be considered to have had overall PD at that point. While it would be ideal to have objective criteria to apply to non-measurable disease, the very nature of that disease makes it impossible to do so; therefore, the increase must be substantial.

### New Lesions

The appearance of new malignant lesions denotes disease progression; therefore, some comments on detection of new lesions are important. There are no specific criteria for the identification of new radiographic lesions; however, the finding of a new lesion should be unequivocal, that is, not attributable to differences in scanning technique, change in imaging modality, or findings thought to represent something other than tumor (for example, some “new” bone lesions may be simply healing or flare of preexisting lesions). This is particularly important when the participant's baseline lesions show PR or CR. For example, necrosis of a liver lesion may be reported on a CT scan report as a “new” cystic lesion, which it is not.

A lesion identified during the study in an anatomical location that was not scanned at baseline is considered a new lesion and will indicate disease progression.

If a new lesion is equivocal, for example because of its small size, continued therapy and follow-up evaluation will clarify if it represents truly new disease. If repeat scans confirm there is definitely a new lesion, then progression should be declared using the date of the initial scan.

### Evaluation of Response

### Timepoint Response (Overall Response)

It is assumed that at each protocol-specified timepoint, a response assessment occurs. Table A provides a summary of the overall response status calculation at each timepoint for participants who have measurable disease at baseline. When participants have non-measurable (therefore non-target) disease only, Table B is to be used.

TABLE A
Timepoint Response: Participants with Target
Lesions (with or without Non-Target Lesions)

| Target | | New | Time point |
|---|---|---|---|
| Lesions | Non-Target Lesions | Lesions | Response |
| CR | CR | No | CR |
| CR | Non-CR/Non-PD or NE | No | PR |
| PR | CR, Non-CR/Non-PD or NE | No | PR |
| SD | CR, Non-CR/Non-PD, or NE | No | SD |
| NE | Non-PD | No | NE |
| PD | Any | Yes or No | PD |
| Any | PD | Yes or No | PD |
| Any | Any | Yes | PD |
| CR | NED ^{b} | No | CR |
| PR | NED ^{b} | No | PR |
| SD | NED ^{b} | No | SD |
| NED ^{a} | Non-CR/Non-PD | No | Non-CR/ |
| | | | Non-PD |
| NED ^{a} | CR | No | CR |
| NED ^{a} | NE | No | NE |
| NED ^{a} | NED ^{b} | No | NED |
| Any non-PD | Any non-PD | NE | NE |

CR = complete response; NE = not evaluable; PD = progressive disease; PR = partial response; SD = stable disease, NED = Non Evaluable Disease.
NED ^{a}: No target lesions identified at Baseline
NED ^{b}: No non-target lesions identified at Baseline

TABLE B
Timepoint Response: Participants with Non-Target Lesions Only

| Non-Target Lesions | New Lesions | Overall Response |
|---|---|---|
| CR | No | CR |
| Non-CR/non-PD | No | Non-CR/non-PD ^{a} |
| Not all evaluated | No | NE |
| Unequivocal PD | Yes or No | PD |
| Any | Yes | PD |

CR = complete response; NE = not evaluable; PD = progressive disease.
^{a} “Non-CR/non-PD” is preferred over “stable disease” for non-target disease since stable disease is increasingly used as an endpoint for assessment of efficacy in some trials; thus, assigning “stable disease” when no lesions can be measured is not advised.

When no imaging/measurement is done at all at a particular timepoint, the participant is not evaluable at that timepoint (see Table C). If only a subset of lesion measurements are made at an assessment, usually the case is also considered not evaluable at that timepoint, unless a convincing argument can be made that the contribution of the individual missing lesion(s) would not change the assigned timepoint response. This would be most likely to happen in the case of PD. For example, if a participant had a baseline sum of 50 mm with three measured lesions and during the study only two lesions were assessed, but those gave a sum of 80 mm, the participant will have achieved PD status, regardless of the contribution of the missing lesion.

If one or more target lesions were not assessed either because the scan was not done or the scan could not be assessed because of poor image quality or obstructed view, the response for target lesions should be “unable to assess” since the participant is not evaluable. Similarly, if one or more non-target lesions are not assessed, the response for non-target lesions should be “unable to assess” except where there is clear progression. Overall response would be “unable to assess” if either the target response or the non-target response is “unable to assess” except where this is clear evidence of progression, as this equates with the case being not evaluable at that timepoint.

Participants with a global deterioration in health status requiring discontinuation of treatment without objective evidence of disease progression at that time should be reported as “symptomatic deterioration.” Every effort should be made to document objective progression even after discontinuation of treatment. Symptomatic deterioration is not a descriptor of an objective response; it is a reason for stopping study therapy.

The objective response status of such participants is to be determined by evaluation of target and non-target lesions as shown in Table A.

For equivocal findings of progression (e.g., very small and uncertain new lesions, cystic changes, or necrosis in existing lesions), treatment may continue until the next scheduled assessment. If at the next scheduled assessment, progression is confirmed, the date of progression should be the earlier date of when progression was suspected.

FDG-PET is not VALIDATED for the use in clinical trials to determine the response but can identify new lesions (Table C) and benefit clinical decisions.

TABLE C
FDG-PET Imaging to Identify New Lesions

| Baseline FDG-PET | Post-baseline FDG-PET | Determination |
|---|---|---|
| Negative FDG-PET | Positive FDG-PET | New lesion (PD) |
| None | Positive FDG-PET | New lesion (PD) |
| | corresponds to a new site of | |
| | disease confirmed by CT/MRI | |
| None | Positive FDG-PET not | Additional follow-up CT/MRI scans |
| | confirmed as a new site of | are needed to determine if there is |
| | disease on CT/MRI | truly progression occurring at that |
| | | site. |
| | | If so: New lesion (PD) with date of |
| | | PD being the date of the initial |
| | | abnormal FDG-PET scan date |
| | | If not: Not a new lesion |
| None | Positive FDG-PET which | Not a new lesion |
| | corresponds to a pre-existing | |
| | site of disease on CT/MRI | |
| | that is not progressing on the | |
| | basis of the anatomic images | |

CT = computed tomography; FDG-PET = fluorodeoxyglucose-positron emission tomography; MRI = magnetic resonance imaging; PD = progressive disease.

TABLE D
Best Overall Response When Confirmation Is Required

| Overall | Overall Response at | |
|---|---|---|
| Response at | Subsequent | |
| First Timepoint | Timepoint | Best Overall Response |
| CR | CR | CR |
| CR | PR | SD, PD, or PR ^{a} |
| CR | SD | SD, provided minimum duration for SD was |
| | | met; otherwise, PD |
| CR | PD | SD, provided minimum duration for SD was |
| | | met; otherwise, PD |
| CR | NE | SD, provided minimum duration for SD was |
| | | met; otherwise, NE |
| PR | CR | PR |
| PR | PR | PR |
| PR | SD | SD |
| PR | PD | SD, provided minimum duration for SD was |
| | | met; otherwise, PD |
| PR | NE | SD, provided minimum duration for SD was |
| | | met; otherwise, NE |
| NE | NE | NE |

CR = complete response; NE = not evaluable; PD = progressive disease; PR = partial response; SD = stable disease.
^{a} If a CR is truly met at the first timepoint, any disease seen at a subsequent timepoint, even disease meeting PR criteria relative to baseline, qualifies as PD at that point (since disease must have reappeared after CR). Best response would depend on whether the minimum duration for SD was met. However, sometimes CR may be claimed when subsequent scans suggest small lesions were likely still present and in fact the participant had PR, not CR, at the first timepoint. Under these circumstances, the original CR should be changed to PR and the best response is PR.

When nodal disease is included in the sum of target lesions and the nodes decrease to “normal” size (<10 mm), they may still have a measurement reported on scans. This measurement should be recorded even though the nodes are normal in order not to overstate progression should it be based on increase in size of the nodes. As noted earlier, this means that participants with CR may not have a total sum of “zero” on the CRF.

Participants with a global deterioration of health status requiring discontinuation of treatment without objective evidence of disease progression at that time should be reported as “symptomatic deterioration.” Every effort should be made to document objective progression even after discontinuation of treatment. Symptomatic deterioration is not a descriptor of an objective response; it is a reason for stopping study therapy. The objective response status of such participants is to be determined by evaluation of target and non-target disease as shown in Tables A-D.

For equivocal findings of progression (e.g., very small and uncertain new lesions; cystic changes or necrosis in existing lesions), treatment may continue until the next scheduled assessment. If at the next scheduled assessment, progression is confirmed, the date of progression should be the earlier date when progression was suspected.

In studies for which participants with advanced disease are eligible (i.e., primary disease still or partially present), the primary tumor should also be captured as a target or non-target lesion, as appropriate. This is to avoid an incorrect assessment of CR if the primary tumor is still present, but not evaluated as a target or non-target lesion.

### 2. INRC

Antitumor activity will be assessed based on clinical and radiographic evidence as described by the INRC (Park et al. 2017, Clin Oncol; 35:2580-7).

At baseline, tumor lesions/lymph nodes will be categorized as measurable or non-measurable as described below. All measurable and non-measurable lesions should be assessed at screening and at subsequent protocol-specified tumor assessment timepoints. Additional assessments may be performed as clinically indicated for suspicion of progression.

Primary and metastatic soft tissue sites will be assessed by measurements in CT/MRI, the Curie score for MIBG scans and/or qualitative scores for FDG-PET scans if the tumor is MIBG non-avid. Bone marrow will be assessed by histology or immunohistochemistry and cytology or immunocytology. Bone marrow with ≤5% tumor involvement will be classified as minimal disease.

Overall response in INRC integrates tumor response in the primary tumor, soft tissue and bone metastases, and bone marrow.

Definition of Target Lesions: At baseline, one primary target lesion (or two in the case of bilateral adrenal disease unless biopsy proves one to be ganglioneuroma) will be selected based on anatomic imaging and radionuclide scans, and will be >10 mm in the Longest Diameter (LD) on CT or MRI and have MIBG uptake for MIBG-avid lesions as shown by the Curie score, or FDG uptake for lesions that are not MIBG-avid.

A maximum of five non-primary target lesions, up to two per organ, are to be chosen to follow throughout the study and for non-nodal lesions will be ≥10 mm or ≥15 mm for nodal lesions in the LD on CT or MRI and must be MIBG (or FDG) avid unless proven to have viable tumor by biopsy.

Definition of Non-Target Lesions: MIBG (or FDG) avid or biopsy proven disease not selected as target disease will be followed as non-target disease and may include disease meeting the size criteria for target disease, in excess of the allowed maximum numbers, non-nodal lesions <10 mm in LD, nodal lesions <15 mm short axis diameter, or non-measurable disease such as leptomeningeal tumor, tumor in cerebrospinal fluid, ascites, or pleural effusion.

At each timepoint, for participants with MIBG avid disease, the reader will evaluate the MIBG scan and provide a semi-quantitative Curie score:

Divides Body into 10 Sectors:
1-9: A score for osteomedullary lesions will be assessed in nine different body regions. Paired segments are considered a single segment (e.g., two femurs make one segment).
10: All extraosseous metastases in the soft tissue throughout the body will be assessed together in one score

### Extension Scale:

A score of 0-3 will be assigned based on the extent of ostemedullary disease in sectors 1-9:
   0=no site per sector
   1=one site per sector
   2=more than one site per sector
   3=massive involvement (>50% of the sector)
A score of 0-3 will be assigned based on the extent of soft tissue lesions in the whole body:
   0=no MIBG-avid soft-tissue disease
   1=a single soft tissue focus
   2=two or more soft-tissue foci
   3=MIBG-avid soft-tissue lesion that occupies more that 50% of the chest or abdomen

For participants that do not have MIBG avid disease, bone disease on FDG-PET will be qualitatively assessed as avid, non-avid, or not evaluable.

PRIMARY (SOFT TISSUE) TUMOR RESPONSE^{a}

| Response | Anatomic + MIBG (FDG-PET ^{b}) imaging |
|---|---|
| Complete Response | <10 mm residual soft tissue at primary site |
| | AND |
| | Complete resolution of MIBG or FDG-PET uptake (for MIBG |
| | non-avid tumors) at primary site |
| Partial Response | ≥30% decrease in longest diameter of primary site |
| | AND |
| | MIBG or FDG-PET uptake at primary site stable, improved, |
| | or resolved |
| Stable Disease | Neither sufficient shrinkage for PR nor sufficient increase for |
| | PD at the primary site |
| Progressive Disease | >20% increase in longest diameter taking as reference the |
| | smallest sum on study (this includes the baseline sum if that |
| | is the smallest on study) |
| | AND |
| | Minimum absolute increase of 5 mm in longest dimension ^{c} |

CR = complete response; FDG-PET = fluorodeoxyglucose-positron emission tomography; MIBG = metaiodobenzylguanidine; PD = progressive disease; PR = partial response; SD = stable disease.
^{a}Not for use in assessment of metastatic sites.
^{b} Used for MIBG non-avid tumors.
^{c} Mass that does not meet PD measurement criteria but has fluctuating MIBG avidity will not be considered PD.

TUMOR RESPONSE AT METASTATIC SOFT TISSUE AND BONE SITES

| Response | Anatomic + MIBG (FDG-PET ^{a}) imaging |
|---|---|
| Complete Response | Resolution of all sites of disease, defined as: |
| | Non-primary target and non-target lesions measure <10 mm |
| | AND |
| | Lymph nodes identified as target lesions decrease to a short |
| | axis <10 mm AND |
| | MIBG uptake or FDG-PET uptake (for MIBG non-avid tumors) |
| | of non-primary lesions resolves completely |
| Partial Response | ≥30% decrease in sum of diameters b of non-primary target |
| | lesions compared with baseline AND all of the following: |
| | Non-target lesions may be stable or smaller in size AND No |
| | new lesions |
| | AND ≥50% reduction in MIBG absolute bone score (relative MIBG |
| | bone score ≥0.1 to ≤0.5) or ≥50% reduction in number of |
| | FDG-PET avid bone lesions ^{b, c} |
| Stable Disease | Neither sufficient shrinkage for PR nor sufficient increase for PD |
| | of non-primary lesions |
| Progressive Disease | Any of the following: |
| | Any new soft tissue lesion detected by CT/MRI that is also |
| | MIBG avid or FDG-PET avid |
| | Any new soft tissue lesion seen on anatomic imaging that is |
| | biopsied and confirmed to be neuroblastoma or |
| | ganglioneuroblastoma |
| | Any new bone site that is MIBG avid |
| | A new bone site that is FDG-PET avid (for MIBG non-avid |
| | tumors) AND has CT/MRI findings consistent with tumor OR |
| | has been confirmed histologically to be neuroblastoma or |
| | ganglioneuroblastoma |
| | >20% increase in longest diameter taking as reference the |
| | smallest sum on study (this includes the baseline sum if that is |
| | the smallest on study) AND minimum absolute increase of 5 |
| | mm in sum of diameters of target soft tissue lesions |
| | Relative MIBG score ≥1.2 ^{d} |

CR = complete response; CT = computed tomography; FDG-PET = fluorodeoxyglucose-positron emission tomography; INRC = International Neuroblastoma Response Criteria; MIBG = metaiodobenzylguanidine; MRI = magnetic resonance imaging; PD = progressive disease; PR = partial response; SD = stable disease.
^{a} Used for MIBG non-avid tumors.
^{b} Sum of diameters is defined as the sum of the short axis of discrete lymph nodes (i.e., cervical, axillary nodes) added to the sum of the longest diameters of non□lymph node soft tissue metastases. Masses of conglomerate non-discrete lymph nodes will be measured using longest diameter.
^{c} For participants with soft tissue metastatic disease, resolution of MIBG and/or FDG-PET uptake at the soft tissue sites is not required; all size reduction criteria must be fulfilled.
^{d} Relative MIBG score is the absolute score for bone lesions at time of response assessment divided by the absolute score for bone lesions at baseline before therapeutic interventions. The same scoring method (e.g., INRC) must be used at all assessment timepoints. MIBG single photon-emission computed tomography (SPECT) or MIBG-SPECT/CT may be used for scoring purposes, but the same imaging methodology should be used for all evaluations.

BONE MARROW METASTASIS RESPONSE ^{a}

| Response | Cytology ^{b}/Histology ^{c} |
|---|---|
| Complete | Bone marrow with no tumor infiltration on reassessment, independent |
| Response | of baseline tumor involvement |
| Minimal Disease | Any of the following: |
| | Bone marrow with ≤5% tumor infiltration and remains >0 to ≤5% |
| | tumor infiltration on reassessment OR |
| | Bone marrow with no tumor infiltration that has ≤5% tumor infiltration |
| | on reassessment OR |
| | Bone marrow with >20% tumor infiltration that has >0 to ≤5% tumor |
| | infiltration on reassessment |
| Stable Disease | Bone marrow with tumor infiltration that remains positive with >5% |
| | tumor infiltration on reassessment but does not meet CR, MD, or PD |
| | criteria |
| Progressive | Any of the following: |
| Disease | Bone marrow without tumor infiltration that becomes >5% tumor |
| | infiltration on reassessment OR |
| | Bone marrow with tumor infiltration that increases by >2-fold and |
| | has >20% tumor infiltration on reassessment |

CR = complete response; MD = minimal disease; PD = progressive disease; SD = stable disease.
Note:
In the case of discrepant results between aspirations or core biopsies from two or more sites taken at the same time, the highest infiltration result should be reported using the criteria in this table.
^{a} Response will be compared with baseline disease evaluation at study enrollment.
^{b} Accompanied by immunocytology (recommended, not mandatory).
^{c} Accompanied by immunohistochemistry; specific recommendations by Burchill et al., Cancer (2016).

DETERMINATION OF OVERALL RESPONSE
Combined Response of Soft Tissue, Bone, and Bone Marrow Disease

| Response | Criterion |
|---|---|
| Complete Response | All components meet criteria for CR |
| Partial Response | PR in at least one component and all other components are |
| | either CR, MD ^{a} (bone marrow), PR (soft tissue or bone), |
| | or NI ^{b}; no component with PD |
| Minimal Response | PR or CR in at least one component but at least one other |
| | component with SD; no component with PD |
| Stable Disease | SD in one component with no better than SD or NI ^{b} in any |
| | other component; no component with PD |
| Progressive Disease | Any component with PD |

CR = complete response; MD = minimal disease; NI = not involved; PD = progressive disease; PR = partial response; SD = stable disease.
^{a} For bone marrow assessment only.
^{b} Site not involved at study entry and remains uninvolved.

### 3. RANO

RANO terminology characterizes lesions as measurable versus non-measurable and target versus non-target in the brain. Measurable lesions are the ones that can be assessed quantitatively. From among the measurable lesions, up to 5 target lesions will be selected at baseline (to be followed through the participant's treatment course). Once a lesion is recorded as a target lesion, it is always a target lesion, even if it falls below the size limits for what is considered measurable at baseline. The remaining lesions (additional measurable and/or non-measurable lesions) are categorized as non-target lesions and do not require measurement, only qualitative assessment. With primary CNS disease, any metastases outside the brain are extremely rare. If this does happen, it will be assessed using RECIST 1.1 (as a non-target lesion at baseline or new lesion post-baseline).

The parameters for image acquisition are to follow the consensus guidelines and standardized Brain Tumor Imaging Protocol (Ellingson et al. 2015, Neuro Oncol; 17:1188-98).

### Response Assessment in Neuro-Oncology Criteria

Antitumor activity will be assessed based on clinical and radiographic evidence as described by the RANO criteria.

All measurable and non-measurable lesions should be assessed using the same techniques as at baseline. Ideally, participants should be imaged on the same MRI scanner, or at least with the same magnet strength, for the duration of the study to reduce difficulties in interpreting changes.

Measurable disease is defined as contrast enhancing lesions with clearly defined margins by MRI scan, with two perpendicular diameters of at least 10 mm, visible on two or more axial slices that are preferably, at most, 5 mm apart with 0-mm skip. If there are multiple contrast-enhancing lesions, up to five of the largest lesions that are suitable for reproducible measurements should be selected as target lesions, and the sum of the products of the perpendicular diameters of these lesions should be determined. If the MRI is performed with thicker slices (>5 mm), the minimum size of a measurable lesion at baseline should be two times the slice thickness. If there are inter-slice gaps, perpendicular diameters must be at least two times the sum of the slice thickness and inter-slice gap for lesions to be considered measurable at baseline. Measurement of tumor around a cyst or surgical cavity represents a particularly difficult challenge. In general, such lesions should be considered non-measurable unless there is a nodular component measuring ≥10 mm in diameter. The cystic or surgical cavity should not be measured in determining response.

Lesions with a necrotic component can be selected as target if they are the only lesion(s) present, otherwise they should be selected as non-target.

Non-measurable disease is defined as either other enhancing measurable lesions, masses with margins not clearly defined (non-enhancing Flair/T2 lesions), or enhancing lesions with maximal perpendicular diameters less than the minimum size for measurability, e.g., 10 mm.

Radiographic response should be determined in comparison with the tumor measurements obtained at pretreatment baseline. The smallest tumor measurements at either pretreatment baseline or after initiation of therapy should be used for determination of progression. Target lesion response categories are defined by changes in enhancing tumor measurements; non-target lesions are assessed qualitatively.

Target lesion, non-target lesion (both enhancing and non-enhancing), and new lesion status are combined as the radiologic (MRI) timepoint response. The MRI timepoint response is combined with clinical status and corticosteroid use to derive the overall response per RANO criteria.

### Target Lesion Response Categories

| Response | Definition |
|---|---|
| CR | Disappearance of all target lesions |
| PR | ≥50% decrease in SPD of longest diameters, taking as reference the |
| | baseline SPD with no evidence of progressive disease. |
| SD | Neither sufficient shrinkage to qualify for PR, nor sufficient increase to |
| | qualify for PD. |
| PD | ≥25% increase in the SPD of the longest diameters, and the greatest |
| | perpendicular diameter of target lesions compared with the smallest |
| | recorded sum (nadir) during the study. |
| NE | One or more target lesions cannot be assessed or accurately determined |
| | or were excised or irradiated, and have not re-appeared or increased. If, in |
| | the event of one or more NE target lesions, the remainder of the evaluable |
| | target lesions compute to a response of PD (25% increase in the SPD of |
| | the longest diameters), then the target response will be PD (not NE). |
| | If there is a change in modalities and the reader is not confident that the |
| | images are truly comparable, or reflects underlying biology, NE should be |
| | selected, unless there is visually clear progression of these lesions. |

CR = Complete Response; NE = Not Evaluable; SD = Stable Disease; SPD = sum of the product; PD = Progressive Disease; PR = Partial Response

### Radiology Lesion Time Point Response Categories

| | Non-Target Lesions | | | | |
|---|---|---|---|---|---|
| | | Non-Contrast | | | MRI Time |
| Target | Contrast | Enhancing | | New | point |
| Lesions | Enhancing (T1) | (FLAIR/T2) | Combined | Lesions | Response |
| CR | Complete | Stable or | CR | No | CR |
| | disappearance | Improved | | | |
| CR | Stable or NE | Stable or | SD | No | PR |
| | | Improved | | | |
| PR | No unequivocal | Stable or | SD | No | PR |
| | progression | Improved | | | |
| SD | No unequivocal | Stable or | SD | No | SD |
| | progression | Improved | | | |
| NE | No unequivocal | Stable or | SD | No | NE |
| | progression | Improved | | | |
| PD | Any | Any | — | Any | PD |
| Any | Unequivocal | Any | PD | Any | PD |
| | progression | | | | |
| Any | Any | Unequivocal | PD | Any | PD |
| | | Progression | | | |
| Any | Any | Any | — | Yes | PD |
| NA* | Complete | Stable or | CR or SD | No | SD |
| | disappearance or | Improved | | | |
| | Stable | | | | |
| NA* | NA** | Not worse | SD | No | NED |

NA* = No target lesions identified at baseline;
NA** = No non-target lesions identified at baseline.
CR = Complete Response; MRI = magnetic resonance imaging; NE = Not Evaluable;
NED = No Evidence of Disease; PD = Progressive Disease; PR = Partial Response; SD = Stable Disease

Measuring Lesions that Split or Coalesce on Treatment

In case lesions coalesce, a plane between them may be maintained that would aid in obtaining maximal diameter measurements of each individual lesion. If the lesions have truly coalesced such that they are no longer separable, the vector of the longest diameter in this instance should be the maximum longest diameter for the coalesced lesion.

Measuring Lesions that Become Too Small to Measure

During the study, all target lesions recorded at baseline should have their actual measurements recorded at each subsequent evaluation, even when very small (e.g., 2 mm). However, sometimes lesions that are recorded as target lesions at baseline become so faint that the radiologist may not feel comfortable assigning an exact measurement and may report them as being too small to measure. When this occurs, it is important that a value be recorded as follows:
If it is the opinion of the radiologist that the lesion has likely disappeared, the measurement should be recorded as 0 mm.
If the lesion is believed to be present and is faintly seen but too small to measure, a default value of 5 mm should be assigned and “too small to measure” should be selected and added to the sum of the SPD.

Increase in corticosteroid dose alone, in the absence of clinical deterioration related to tumor, will not be used as a determinant of progression. Participants with stable imaging studies whose corticosteroid dose was increased for reasons other than clinical deterioration related to tumor do not qualify for stable disease or progression. They should be observed closely. If their corticosteroid dose can be reduced back to baseline, they will be considered as having stable disease; if further clinical deterioration related to tumor becomes apparent, they will be considered to have progression. The date of progression should be the first timepoint at which corticosteroid increase was necessary.

The definition of clinical deterioration is left to the discretion of the treating physician, but it is recommended that a decline in the Karnofsky or Lansky Performance Status from 100 or 90 to 70 or less, a decline in Performance Status of at least 20 from 80 or less, or a decline in Performance Status from any baseline to 50 or less, for at least 7 days, be considered neurologic deterioration unless attributable to comorbid events or changes in corticosteroid dose.

Participants with non-measurable enhancing disease whose lesions have significantly increased in size and become measurable (minimal bidirectional diameter of ≥10 mm and visible on at least two axial slices that are preferably, at most, 5 mm apart with
0-mm skip) will also be considered to have experienced progression. The transition from a non-measurable lesion to a measurable lesion resulting in progression can theoretically occur with relatively small increases in tumor size (e.g., a 9×9 mm lesion [non-measurable] increasing to a 10×11 mm lesion [measurable]). Ideally, the change should be significant (>5 mm increase in maximal diameter or >25% increase in the sum of the products of perpendicular diameters of enhancing lesions).

In general, if there is doubt about whether the lesion has progressed, continued treatment and close follow-up evaluation will help clarify whether there is true progression. If there is uncertainty regarding whether there is progression, the participant may continue on treatment and remain under close observation (e.g., evaluated at 4-week intervals). If subsequent evaluations suggest that the participant is in fact experiencing progression, then the date of progression should be the timepoint at which this issue was first raised.

### RANO Response

| Response | Definition |
|---|---|
| Complete | Requires all of the following: |
| response | Confirmed CR on MRI (response sustained for at least 4 weeks |
| | with no signs of progression) |
| | Participants must be off corticosteroids (or on physiological |
| | replacement doses only) |
| | Clinical status is stable or improved |
| | Note: Participants with non-measurable disease at baseline only cannot |
| | have a complete response; the best response possible is stable disease. |
| Partial response | Requires all of the following: |
| | Confirmed PR on MRI (response sustained for at least 4 weeks |
| | with no signs of progression) |
| | Same or lower dose of corticosteroids compared with baseline |
| | Clinical status is stable or improved |
| | Note: Participants with non-measurable disease only cannot have a |
| | partial response; the best response possible is stable disease. |
| Stable disease | Requires all of the following: |
| | SD on MRI |
| | Clinical status is stable or improved |
| | Note: In the event that the corticosteroid dose was increased for new |
| | symptoms and signs without confirmation of disease progression on |
| | neuroimaging, and subsequent follow-up imaging shows that this |
| | increase in corticosteroids was required because of disease |
| | progression, the last scan considered to show stable disease will be the |
| | scan obtained when the corticosteroid dose was equivalent to the |
| | baseline dose. |
| Progression | Defined by any of the following: |
| | PD on MRI |
| | Clear clinical deterioration not attributable to other causes apart |
| | from the tumor (e.g., seizures, medication adverse effects, |
| | complications of therapy, cerebrovascular events, infection, |
| | and so on) or changes in corticosteroid dose |
| | Failure to return for evaluation due to death or deteriorating |
| | condition |

CR = Complete Response, FLAIR = fluid-attenuated inversion recovery; MRI = magnetic resonance imaging; PR = Partial Response, SD = Stable Disease, PD = Progressive Disease

### Laboratory, Biomarker, Pharmacokinetic and Other Biological Samples

Blood for determination of plasma concentrations of alectinib, and its metabolite M4 will be collected.

Samples for the following biomarker assessments will be collected. Exploratory biomarker research may include, but will not be limited to, analysis of somatic mutations and gene signatures associated with disease type (i.e., ALK fusion-positive solid or CNS tumours), as well as evaluation of candidate biomarkers of response to alectinib (i.e., ALK).

Single ECG recordings will be obtained at specified timepoints.

Biomarkers of bone formation and resorption and biomarkers of calcium metabolism and skeletal homeostasis will be measured from blood (subject to blood volume limitations). These biomarkers include: bone-specific ALP, Vitamin D, and parathyroid hormone.

Bone markers will be analyzed at baseline (up to two baseline assessments due to variability), 6 weeks (on Day 15 of Cycle 2), after Cycle 6 (Day 1 of Cycle 7) and every 6 cycles thereafter (paired with the DXA scan at 12 cycles if possible) until the follow-up visit.

Serial DXA scans will be performed in participants at baseline, after Cycle 6 (Day 1 of Cycle 7), every 12 cycles until the end of treatment, and 30 days after the final dose, whichever occurs last.

Serial X-rays will be performed in participants on treatment at baseline, at 6 weeks, Cycle 6 and, every 6 months until the end of treatment.

### Clinical Outcome Assessments

Patient-reported outcome (PRO) and observer-reported outcome (ObsRO) instruments completed by the participant and participants' caregiver, respectively will be used to assess the treatment tolerability of alectinib. In addition, PRO instruments will enable the capture of each participant's direct experience with alectinib.

PRO data will be collected through use of the following instruments: Paediatric PRO-CTCAE and medication Acceptability Survey. ObsRO data will be collected through use of the Caregiver (Proxy) PRO-CTCAE.

To evaluate the neuro-psychological function (i.e., IQ) of participants, the Bayley or Wechsler scale (adapted for age) should be administered by a neuropsychologist or appropriately qualified professional and according to local practice.

To better understand the acceptability and palatability of alectinib, a survey will be administered immediately following administration of alectinib on Day 1 of Cycle 1.

### Adverse Events

The adverse event severity grading scale for the NCI CTCAE (v5.0) will be used for assessing adverse event severity. Table 6 will be used for assessing severity for adverse events that are not specifically listed in the NCI CTCAE.

TABLE 6
Adverse Event Severity Grading Scale for Events
Not Specifically Listed in NCI CTCAE

| Grade | Severity |
|---|---|
| 1 | Mild; asymptomatic or mild symptoms; clinical or diagnostic observations |
| | only; or intervention not indicated |
| 2 | Moderate; minimal, local, or non-invasive intervention indicated; or limiting |
| | age appropriate- instrumental activities of daily living ^{a} |
| 3 | Severe or medically significant, but not immediately life threatening-; |
| | hospitalization or prolongation of hospitalization indicated; disabling; or |
| | limiting self-care activities of daily living ^{b, c} |
| 4 | Life-threatening consequences or urgent intervention indicated ^{d} |
| 5 | Death related to adverse event ^{d} |

NCI CTCAE = National Cancer Institute Common Terminology Criteria for Adverse Events.
Note:
Based on the most recent version of NCI CTCAE (v5.0), which can be found at: http://ctep.cancer.gov/protocolDevelopment/electronic_applications/ctc.htm
^{a} Instrumental activities of daily living refer to preparing meals, shopping for groceries or clothes, using the telephone, managing money, etc.
^{b} Examples of self-care activities of daily living include bathing, dressing and undressing, feeding oneself, using the toilet, and taking medications, as performed by participants who are not bedridden.
^{c} If an event is assessed as a “significant medical event,” it must be reported as a serious adverse event.
^{d} Grade 4 and 5 events must be reported as serious adverse events.

Investigators should use their knowledge of the participant, the circumstances surrounding the event, and an evaluation of any potential alternative causes to determine whether an adverse event is considered to be related to the study drug, indicating “yes” or “no” accordingly. The following guidance should be taken into consideration:

### Temporal Relationship of Event Onset to the Initiation of Study Drug

Course of the event, with special consideration of the effects of dose reduction, discontinuation of study drug, or reintroduction of study drug (as applicable)
Known association of the event with the study drug or with similar treatments
Known association of the event with the disease under study
Presence of risk factors in the participant or use of concomitant medications known to increase the occurrence of the event
Presence of non-treatment-related factors that are known to be associated with the occurrence of the event

### Statistical Considerations and Analysis Plan

The analysis populations are defined for this study below:
The intent-to-treat (ITT) population is defined as all enrolled participants regardless of whether they were dosed.
The efficacy-evaluable population is defined as all participants enrolled at the paediatric RP2D with measurable disease who have received at least one dose of study drug and have tumours with confirmed ALK fusion.
The safety-evaluable population is defined as all enrolled participants who have received any amount of study treatment.
DLT-evaluable population is defined as all participants enrolled in Part 1 who either have completed at least 80% of the prescribed dose of alectinib in Cycle 1 or have experienced a DLT in Cycle 1.
PK-evaluable population is defined as all participants who were administered alectinib and have at least one timepoint with a measurable concentration; data from this population will be included in the PK analysis dataset. Participants will be excluded from the PK analysis population if they significantly violate the inclusion or exclusion criteria, deviate significantly from the protocol or if data are unavailable or incomplete which may influence the PK analysis. Excluded cases will be documented together with the reason for exclusion.
PRO-evaluable population is defined as all participants who have received at least one dose of study drug and have one baseline and at least one post-baseline measurement.

### Determination of Sample Size

The safety run-in phase of this study will enroll a minimum of 6 participants and until approximately 12 participants to confirm the paediatric RP2D. Explicit power and type I error considerations are not factored into the design of the paediatric RP2D determination, as this phase is designed to obtain preliminary safety and PK information on the study drug.

An initial response assessment of participants enrolled solely in Part 2 and a subsequent additional response assessment of participants enrolled in both Parts 2 and 3 are planned for the expansion stage in order to make a preliminary assessment of the efficacy of the study drug. Taking into account the historical control ORR of 30%, a minimum of 4 responders among 10 participants (40%) in Part 2 will be required for advancement to the additional expansion in Part 3. For initial efficacy assessment, if the true ORR is 20% (within 10% non-inferiority margin of reference ORR of 30%), the false positive probability (type I error) is 12% to observe an ORR of ≥40% in 10 participants.

The interim analysis is for futility purpose only and the trial will not stop if efficacy is observed at the interim analysis. Part 3 will enroll a minimum of an additional 14 participants. Participants included in the safety run-in phase will not be included in the initial response assessment of the participants in Part 2.

The primary efficacy analysis will include all participants enrolled at the paediatric RP2D who receive any amount of study drug with confirmed ALK-fusion. The primary efficacy analysis will occur after these participants have been followed for at least 6 months. A minimum number of 10 participants with solid tumours and 10 participants with CNS tumours will be enrolled in this trial. With at least 30 participants (i.e., at least 6 participants enrolled at the paediatric RP2D in the safety run-in phase and a total of 24 participants enrolled in the initial and additional response assessment) and, if expanded to Part 3, this trial will be able to detect a large benefit conferred by alectinib in terms of the ORR. For example, an observed response rate of 50% in 30 participants (i.e., 15 responders) with ALK-fusion positive solid or CNS tumours will have a 95% CI, excluding an ORR of 30% with 90% power if the true ORR is 60%. The primary efficacy analysis will require centrally confirmed blinded tumour assessments by the BICR.

### Demographic and Baseline Characteristics

Demographic and baseline characteristics (including age, sex, and disease characteristics) will be summarized in the ITT population using means, standard deviations, medians, and ranges for continuous variables and proportions for categorical variables, as appropriate.

### Safety Analyses

Safety will be characterized by incidence of adverse events and DLTs, as well as change from baseline in laboratory test results, vital signs and ECGs. Study treatment exposure (such as treatment duration, total dose received, and number of cycles and dose modifications) will be summarized with descriptive statistics.

The incidence and severity of DLTs assessed during the first cycle of study treatment will be summarized using descriptive statistics. DLT analyses will be based on the DLT-evaluable population.

### Exploratory Analyses of PRO-CTCAE Data

Paediatric and Caregiver (Proxy) PRO-CTCAE analyses will be primarily descriptive, with a focus on characterizing the pattern of symptomatic treatment toxicities over the course of the study. Results from these exploratory analyses will be presented separately from the other safety analyses. Paediatric and Caregiver (Proxy) PRO-CTCAE data will be analyzed at the item level consistent with current NCI recommendations for data handling (Basch et al. 2014). The analysis population will be the safety-evaluable population.

### Exploratory Analyses of Growth and Development Data

SDS growth data from WHO and CDC will be used as reference per participant's age and gender.

### Pharmacokinetic Analyses

All participants who receive alectinib and have at least one timepoint with a measurable concentration will be included in the PK-evaluable dataset. Participants will be excluded from the PK analysis population if they significantly violate the inclusion or exclusion criteria, deviate significantly from the protocol or if data are unavailable or incomplete which may influence the PK analysis. Excluded cases will be documented together with the reason for exclusion.

Individual and mean plasma alectinib and M4 concentration versus time data will be tabulated and plotted by dose level. The plasma pharmacokinetics of alectinib and M4 will be summarized (e.g., area under the concentration-time curve [AUC]), Cₘₐₓ, Tₘₐₓ, and C_{trough} as appropriate), and if permitted for data collected, grouped according to treatment received. Alectinib and M4 PK parameters will also be tabulated and summarized (mean, standard deviation, coefficient of variation, median, minimum, and maximum as applicable). Inter-participant variability and drug accumulation will be evaluated if permitted.

Non-linear mixed effects modeling (software NONMEM®) will be used to analyze the PK concentrations. Population and individual PK parameters will be estimated and the influence of various covariates (e.g., age, gender and body weight) on these parameters will be investigated in an exploratory way. Data may be pooled with data from other studies with alectinib in order to improve the parameter estimates from the model.

Secondary PK parameters (e.g., Cₘₐₓ and AUC) may be derived from the model for each individual included in the PK analysis and will be presented descriptively.

### Efficacy Analyses

The analysis population for the efficacy analyses will be efficacy evaluable participants defined as all participants enrolled at the paediatric RP2D with measurable disease who have received at least one dose of study drug and have tumours with confirmed ALK fusion, unless otherwise specified.

### Primary Efficacy Endpoint: Objective Response Rate

The primary efficacy endpoint is ORR in participants treated at the paediatric RP2D, as assessed by BICR, defined as the proportion of participants with an objective response. The objective response for individual participants will be assessed per RECIST v1.1 for participants with extracranial tumours, and per RANO for participants with primary CNS tumours, and INRC for neuroblastoma. An objective response is defined as a CR or PR per disease-specific response criteria, confirmed ≥4 weeks apart in two separate tumour assessments. Participants not meeting these criteria (including participants without a postbaseline tumour assessment) will be considered non-responders.

An estimate of the ORR and its 95% CI, estimated using the Clopper-Pearson method will be calculated for comparison with a 30% threshold. If the lower bound of the 95% CI exceeds 30%, alectinib will be considered of therapeutic interest in ALK fusion-positive tumours.

The analysis will occur at the following timepoints:
At initial efficacy assessment (after part 2 have completed) when a minimum of 10 paediatric participants have been enrolled and followed for at least 6 months
If conducted, at the time of final efficacy assessment (after part 3 completed), when a minimum of 30 efficacy-evaluable participants, including at least a minimum number of 10 participants with solid tumour and 10 participants with CNS tumour, who have been followed for at least 6 months

### Secondary Efficacy Endpoints

The secondary efficacy endpoints for all participants, as assessed by BICR and investigators, are defined above, including TTR, CBR, PFS, OS and investigator-assessed ORR in the paediatric RP2D population; DOR in participants who achieve an objective response. The Kaplan-Meier approach will be used to estimate median for all time-to-event endpoints including PFS, TTR, DOR and OS. The 95% CI of the median will be estimated using Brookmeyer and Crowley method.

For calculation of PFS and TTR, data for participants without disease progression or death will be censored at the date of the last tumour assessment at which the participant was known to be progression-free or, if no tumour assessment is performed after the baseline visit, at the date of initiation of study drug.

DOR will be calculated in participants who achieve an objective response, data for participants without disease progression or death by the date of data cutoff for analysis will be censored at the date of the last tumour assessment. If no tumour assessments were performed after the date of the first occurrence of objective response, DOR will be censored at the date of the first occurrence of objective response plus 1 day.

For OS, data for participants who are not reported as having died at the time of analysis will be censored at the date when they were last known to be alive.

Furthermore, to assess the consistency of study results in two subgroups defined by CNS tumours and solid tumours ORR, CBR, TTR, OS, and DOR in these subgroups will be examined. ORR and its 95% CI will be summarized (where applicable) and listed for participants with solid tumours and participants with CNS tumours. DOR will be listed for the responders in the two subgroups.

### Exploratory Efficacy Endpoints

To assess the consistency of study results in subgroups defined by tumour types ORR, CBR, TTR, OS, and DOR in these subgroups will be examined. ORR and its 95% CI will be summarized and listed for participants for participants with each tumour type. DOR will be listed for the responders in the available tumour types.

### Exploratory Biomarker Analyses

Although no formal statistical analysis of exploratory biomarkers will be performed, data may be analyzed in the context of this study and in aggregate with data from other studies.

### Results

Patients with ALK fusion-positive solid or CNS tumours for whom prior treatment has proven to be ineffective or for whom there is no satisfactory treatment available, less than 18 years of age, were eligible. Patients were recruited to the Part 1, safety run-in to confirm the recommended phase 2 dose and monitor drug pharmacokinetics. Investigators reported responses according to RANO (CNS tumours), RECIST (solid tumours) or INRC (neuroblastoma) criteria with a data cut off of Jun. 12, 2023.

In total 8 patients at a median age of 11 years (range 6 months-17 years), diagnosed with inflammatory myofibroblastic tumour (n=2), high grade glioma (n=3), renal cell carcinoma (n=2), and anaplastic large cell lymphoma (n=1, protocol deviation) were enrolled (Tables 7 and 8).

Among 7 patients evaluable for efficacy, 6 had not received prior systemic therapy. ALK fusion partners were KIF5C in 2 patients and TPM3, CLTC, PLEKHA7, DCTN1 and HNRNPA3 in 1 patient each (Table 11).

Median treatment duration was 12 months (range 1-21).

No Dose Limiting Toxicities were reported. Seven patients experienced a related Adverse Event (AE) including 2 patients with Grade ≥3 related AE. There were no AE-related deaths, and no new safety signals detected.

Investigators reported Best Overall Responses as follows: in 4 patients (57.1%; 1 CR, 3 PR), 2 patients (28.6%) with Stable Disease and 1 patient had not undergone a tumour assessment at the time of data cut off (Table 10).

TABLE 7
Demographics and baseline characteristics.
Alectinib (N = 8)

| | | | | | |
|---|---|---|---|---|---|
| Age (yr) | Mean (SD) | 10.5 (6.2) | Weight (kg) | Mean (SD) | 41.62 (26.77) |
| | Median | 11.0 | at baseline | Median | 43.10 |
| | Min-Max | 0-17 | | Min-Max | 7.1-78.5 |
| Age at | <2 years | 1 (12.5%) | Type of | Surgery | 5 (62.5%) |
| baseline | 2-<6 years | 1 (12.5%) | Prior Anti- | Chemotherapy | 2 (25%) |
| | 6-<12 years | 2 (25.0%) | Cancer | Radiotherapy | 1 (12.5%) |
| | 12-<18 years | 4 (50.0%) | Therapy | | |
| Sex | Male | 4 (50.0%) | Number of | 0 lines | 6 (75%) |
| | Female | 4 (50.0%) | Prior Lines | 1 line | 2 (25%) |
| | | | of Therapy | | |
| Ethnicity | Hispanic or | 2 (25.0%) | ALK Fusion | KIF5C | 2 (25.0%) |
| | Latino | | Partner | | |
| | Not Hispanic | 6 (75.0%) | | TPM3, CLTC, | 1 (12.5%) |
| | or Latino | | | NPM, | |
| | | | | PLEKHA7, | |
| | | | | DCTN1, | |
| | | | | HNRNPA3 | |
| Race | American | 1 (12.5%) | Tumour | Primary | 5 (62.5%) |
| | Indian or | | Type | Tumour | |
| | Alaska | | | | |
| | Native | | | | |
| | Asian | 2 (25.0%) | | Metastatic | 2 (25.0%) |
| | | | | Tumour | |
| | White | 5 (62.5%) | | Locally | 1 (12.5%) |
| | | | | Recurrent | |
| | | | | Cancer | |

TABLE 8
Patient demographics and baseline characteristics

| | | | Body | |
|---|---|---|---|---|
| Patient ID | Age | Sex | Weight | Tumour Type |

| | | | | | |
|---|---|---|---|---|---|
| 1 | 16 | yr | M | 61 kg | L metastatic renal |
| | | | | | carcinoma |
| 2 | 10 | yr | F | 25.5 kg | IMT |
| 3 | 6 | mo | F | ~7 kg | HGG |
| 4 | 12 | yr | F | 61.5 kg | ALCL¹ |
| 5 | 5 | yr | F | 16.7 kg | HGG |
| 6 | 17 | yr | M | 78.5 kg | R metastatic renal |
| | | | | | carcinoma |
| 7 | 17 | yr | M | 61.0 kg | IMT of the lung |
| 8 | 7 | yr | M | 21.5 kg | HGG |

¹Pt. 4's enrolment was a protocol deviation because ALCL was incorrectly classified as a solid tumour.
yr: years; mo: months; L: left; R: right; IMT: inflammatory Myofibroblastic Tumour; HGG: High Grade Glioma; ALCL: Anaplastic Large Cell Lymphoma

TABLE 9
Exposure summary

| Exposure Parameters | Number of Patients (n = 8) |
|---|---|
| Treatment duration (days) | |
| n | 8 |
| Mean (SD) | 310.5 (240.0) |
| Median | 352.0 |
| Min-Max | 29-637 |
| Total cumulative dose (mg) | |
| n | 8 |
| Mean (SD) | 217228.00 (258910.33) |
| Median | 100812.00 |
| Min-Max | 7200.0-764400.0 |
| Number of doses | |
| n | 8 |
| Mean (SD) | 556.5 (505.8) |
| Median | 497.0 |
| Min-Max | 12-1274 |
| Number of cycles | |
| n | 8 |
| Mean (SD) | 11.1 (8.1) |
| Median | 11.5 |
| Min-Max | 2-23 |

TABLE 10
Overall efficacy summary

| | Best Overall Response |
|---|---|
| Response Category | (Investigator, %) |
| Total number of patients | N = 8 |
| Total Responders | 5 (62.5%) |
| Complete Response (CR) | 1 (12.5%) |
| Partial Response (PR) | 4 (50.0%) |
| Stable Disease (SD) | 2 (25.0%) |
| Progressive Disease (PD) | 0 (0.0%) |
| Missing/Not evaluable¹ | 1 (12.5%) |

¹Response assessments for 1 patient not completed at the time of the data cut.

TABLE 11
Part 1 Dose Confirmation. Clinical efficacy summary (n = 8).

| | | | Dose | | Confirmed | |
|---|---|---|---|---|---|---|
| | | | assigned at | BOR⁴ by | DOR⁵ by | Time to |
| Patient | ALK fusion | | enrolment | Investigator | investigator | response |
| ID | partner | Formulation | (mg, BID) | (BICR)² | (BICR)² | (months) |
| 1 | TPM3 | Capsule | 600 | PR (—) | — (—) | — |
| 2 | CLTC | Capsule | 300 | SD (—) | — (—) | — |
| 3 | KIF5C | Suspension | 60 | CR (—) | — (—) | — |
| 4¹ | NPM | Capsule | 600 | PR (—) | — (—) | — |
| 5 | KIF5C | Capsule | 230* | SD (—) | — (—) | — |
| 6 | PLEKHA7 | Capsule | 600 | PR (—) | — (—) | — |
| 7 | DCTN1 | Capsule | 600 | PR³ (—) | — (—) | — |
| 8 | HNRNPA3 | Capsule | 270 | NE (—) | — (—) | — |

¹Pt. 4's enrolment was a protocol deviation because ALCL was incorrectly classified as a solid tumour and will not be included in efficacy analyses.
²BICR has not been completed at time of data cut.
³Unconfirmed investigator assessed response.
⁴BOR = Best overall response.
⁵DOR = duration of response.
*Manually corrected number due to confirmed data entry error.

### CONCLUSIONS

Alectinib was generally well tolerated in paediatric patients with ALK-fusion positive solid or CNS tumours. In this hard-to-treat population, efficacy results are promising with the majority of patients experiencing a response, demonstrating a favourable benefit-risk profile.

1. A method of treating an anaplastic lymphoma kinase (ALK) fusion-positive solid tumour or central nervous system tumour, comprising administering to a subject in need of such treatment a therapeutically effective amount of alectinib, or a pharmaceutically acceptable salt thereof, wherein the subject is aged <18 years.

2. The method according to claim 1, wherein the ALK fusion-positive central nervous system tumour is a non-metastatic or primary central nervous system tumour.

3. The method according to claim 1 or claim 2, wherein the ALK fusion-positive solid tumour or central nervous system tumour is a neuroblastoma, renal cell carcinoma, papillary or medullary thyroid cancer, skin tumour, such as Spitz tumour or melanoma, inflammatory myofibroblastic tumour, soft tissue sarcoma, such as undifferentiated or rhabdomyosarcoma, osteosarcoma, malignant peripheral nerve sheath tumour, anaplastic large cell lymphoma, ganglioglioma, or glioma (low- or high-grade).

4. The method according to claim 3, wherein the ALK fusion-positive solid tumour or central nervous system tumour is an inflammatory myofibroblastic tumour, high-grade glioma, renal cell carcinoma, or anaplastic large cell lymphoma.

5. The method according to claim 4, wherein the ALK fusion-positive solid tumour or central nervous system tumour is an inflammatory myofibroblastic tumour or high-grade glioma.

6. The method according to any of claims 1 to 5, wherein the ALK fusion-positive solid tumour or central nervous system tumour comprises a mutation wherein ALK is fused to KIFC, TPM3, CLTC, PLEKHA7, DCTN1 or HNRNPA3.

7. The method according to any of claims 1 to 6, wherein the subject is aged <2 years.

8. The method according to any of claims 1 to 6, wherein the subject is aged ≥2 to <6 years.

9. The method according to any of claims 1 to 6, wherein the subject is aged ≥6 to <12 years.

10. The method according to any of claims 1 to 6, wherein the subject is aged ≥12 to <18 years.

11. The method according to any preceding claim, wherein the subject does not comprise a mutation conferring resistance to ALK inhibitors.

12. The method according to any preceding claim, wherein the subject has not had prior exposure to an ALK inhibitor.

13. The method according to any preceding claim, wherein the subject has had prior treatment for an ALK fusion-positive solid tumour or central nervous system tumour.

14. The method according to any preceding claim, wherein the alectinib, or a pharmaceutically acceptable salt thereof, is administered orally.

15. The method according to any preceding claim, wherein the alectinib, or a pharmaceutically acceptable salt thereof, is administered twice daily.

16. The method according to any preceding claim, wherein the total daily dose of alectinib, or a pharmaceutically acceptable salt thereof, is between 50 and 1500 mg, preferably wherein:
i. the subject is ≥40 kg and the total daily dose of alectinib, or pharmaceutically acceptable salt thereof, is between 600 and 1500 mg;
ii. the subject is ≥35 to <40 kg and the total daily dose of alectinib, or pharmaceutically acceptable salt thereof, is between 400 and 1000 mg;
iii. the subject is ≥30 to <35 kg and the total daily dose of alectinib, or pharmaceutically acceptable salt thereof, is between 300 and 800 mg;
iv. the subject is ≥25 to <30 kg and the total daily dose of alectinib, or pharmaceutically acceptable salt thereof, is between 300 and 700 mg;
v. the subject is ≥20 to <25 kg and the total daily dose of alectinib, or pharmaceutically acceptable salt thereof, is between 250 and 600 mg;
vi. the subject is ≥15 to <20 kg and the total daily dose of alectinib, or pharmaceutically acceptable salt thereof, is between 200 and 500 mg;
vii. the subject is ≥10 to <15 kg and the total daily dose of alectinib, or pharmaceutically acceptable salt thereof, is between 150 and 400 mg;
viii. the subject is ≥7.5 to <10 kg and the total daily dose of alectinib, or pharmaceutically acceptable salt thereof, is between 100 and 300 mg;
ix. the subject is ≥5 to <7.5 kg and the total daily dose of alectinib, or pharmaceutically acceptable salt thereof, is between 75 and 250 mg; or
x. the subject is ≥3.5 to <5 kg and the total daily dose of alectinib, or pharmaceutically acceptable salt thereof, is between 50 and 200 mg.

17. The method according to any preceding claim, wherein the alectinib, or pharmaceutically acceptable salt thereof, is administered in a pharmaceutical composition.

18. The method according to claim 17, wherein the pharmaceutical composition comprises alectinib hydrochloride.

19. The method according to claim 17 or claim 18, wherein the pharmaceutical composition is formulated as a capsule, preferably a hard capsule.

20. The method according to claim 19, wherein the capsule comprises alectinib hydrochloride equivalent to 20 mg or 150 mg alectinib as free base.

21. The method according to claim 19 or claim 20, wherein:
i. the subject is ≥40 kg and the total daily dose of alectinib, or pharmaceutically acceptable salt thereof, is between 1000 and 1400 mg, preferably the total daily dose is 1200 mg, even more preferably 600 mg BID;
ii. the subject is ≥35 to <40 kg and the total daily dose of alectinib, or pharmaceutically acceptable salt thereof, is between 700 and 900 mg, preferably the total daily dose is 760 mg, even more preferably 380 mg BID;
iii. the subject is ≥30 to <35 kg and the total daily dose of alectinib, or pharmaceutically acceptable salt thereof, is between 600 and 800 mg, preferably the total daily dose is 680 mg, even more preferably 340 mg BID;
iv. the subject is ≥25 to <30 kg and the total daily dose of alectinib, or pharmaceutically acceptable salt thereof, is between 500 and 700 mg, preferably the total daily dose is 600 mg, even more preferably 300 mg BID;
v. the subject is ≥20 to <25 kg and the total daily dose of alectinib, or pharmaceutically acceptable salt thereof, is between 500 and 600 mg, preferably the total daily dose is 540 mg, even more preferably 270 mg BID;
vi. the subject is ≥15 to <20 kg and the total daily dose of alectinib, or pharmaceutically acceptable salt thereof, is between 400 and 500 mg, preferably the total daily dose is 460 mg, even more preferably 230 mg BID;
vii. the subject is ≥10 to <15 kg and the total daily dose of alectinib, or pharmaceutically acceptable salt thereof, is between 300 and 400 mg, preferably the total daily dose is 380 mg, even more preferably 190 mg BID;
viii. the subject is ≥7.5 to <10 kg and the total daily dose of alectinib, or pharmaceutically acceptable salt thereof, is between 200 and 300 mg, preferably the total daily dose is 280 mg, even more preferably 140 mg BID;
ix. the subject is ≥5 to <7.5 kg and the total daily dose of alectinib, or pharmaceutically acceptable salt thereof, is between 150 and 250 mg, preferably the total daily dose is 200 mg, even more preferably 100 mg BID; or
x. the subject is ≥3.5 to <5 kg and the total daily dose of alectinib, or pharmaceutically acceptable salt thereof, is between 100 and 200 mg, preferably the total daily dose is 160 mg, even more preferably 80 mg BID.

22. The method according to claim 19 or claim 20, wherein the pharmaceutical composition is formulated as a suspension.

23. The method according to claim 22, wherein:
i. the subject is ≥40 kg and the total daily dose of alectinib, or pharmaceutically acceptable salt thereof, is between 600 and 800 mg, preferably the total daily dose is 718 mg, even more preferably 359 mg BID;
ii. the subject is ≥35 to <40 kg and the total daily dose of alectinib, or pharmaceutically acceptable salt thereof, is between 400 and 500 mg, preferably the total daily dose is 456 mg, even more preferably 228 mg BID;
iii. the subject is ≥30 to <35 kg and the total daily dose of alectinib, or pharmaceutically acceptable salt thereof, is between 350 and 450 mg, preferably the total daily dose is 408 mg, even more preferably 204 mg BID;
iv. the subject is ≥25 to <30 kg and the total daily dose of alectinib, or pharmaceutically acceptable salt thereof, is between 300 and 400 mg, preferably the total daily dose is 360 mg, even more preferably 180 mg BID;
v. the subject is ≥20 to <25 kg and the total daily dose of alectinib, or pharmaceutically acceptable salt thereof, is between 280 and 380 mg, preferably the total daily dose is 324 mg, even more preferably 162 mg BID;
vi. the subject is ≥15 to <20 kg and the total daily dose of alectinib, or pharmaceutically acceptable salt thereof, is between 220 and 320 mg, preferably the total daily dose is 276 mg, even more preferably 138 mg BID;
vii. the subject is ≥10 to <15 kg and the total daily dose of alectinib, or pharmaceutically acceptable salt thereof, is between 180 and 280 mg, preferably the total daily dose is 228 mg, even more preferably 114 mg BID;
viii. the subject is ≥7.5 to <10 kg and the total daily dose of alectinib, or pharmaceutically acceptable salt thereof, is between 100 and 200 mg, preferably the total daily dose is 168 mg, even more preferably 84 mg BID;
ix. the subject is ≥5 to <7.5 kg and the total daily dose of alectinib, or pharmaceutically acceptable salt thereof, is between 80 and 180 mg, preferably the total daily dose is 120 mg, even more preferably 60 mg BID; or
x. the subject is ≥3.5 to <5 kg and the total daily dose of alectinib, or pharmaceutically acceptable salt thereof, is between 50 and 150 mg, preferably the total daily dose is 96 mg, even more preferably 48 mg BID.

24. Alectinib, or a pharmaceutically acceptable salt thereof, for use in a method of treating an ALK fusion-positive solid tumour or central nervous system tumour in a subject, wherein the subject is aged <18 years.

25. The alectinib, or pharmaceutically acceptable salt thereof, for use according to claim 23, wherein the alectinib, or a pharmaceutically acceptable salt thereof, is administered in a pharmaceutical composition.

26. The alectinib, or pharmaceutically acceptable salt thereof, or pharmaceutical composition for use according to claim 24 or claim 25, wherein the ALK fusion-positive central nervous system tumour is a non-metastatic or primary central nervous system tumour.

27. The alectinib, pharmaceutically acceptable salt thereof, or pharmaceutical composition for use according to any of claims 24 to 26, wherein the ALK fusion-positive solid tumour or central nervous system tumour is a neuroblastoma, renal cell carcinoma, papillary or medullary thyroid cancer, skin tumour, such as Spitz tumour or melanoma, inflammatory myofibroblastic tumour, soft tissue sarcoma, such as undifferentiated or rhabdomyosarcoma, osteosarcoma, malignant peripheral nerve sheath tumour, anaplastic large cell lymphoma, ganglioglioma, or glioma (low- or high-grade).

28. The alectinib, pharmaceutically acceptable salt thereof, or pharmaceutical composition for use according to claim 27, wherein the ALK fusion-positive solid tumour or central nervous system tumour is an inflammatory myofibroblastic tumour, high-grade glioma, renal cell carcinoma, or anaplastic large cell lymphoma.

29. The alectinib, pharmaceutically acceptable salt thereof, or pharmaceutical composition for use according to claim 28, wherein the ALK fusion-positive solid tumour or central nervous system tumour is an inflammatory myofibroblastic tumour or high-grade glioma.

30. The alectinib, pharmaceutically acceptable salt thereof, or pharmaceutical composition for use according to any of claims 24 to 29, wherein the ALK fusion-positive solid tumour or central nervous system tumour comprises a mutation wherein ALK is fused to KIFC, TPM3, CLTC, PLEKHA7, DCTN1 or HNRNPA3.

31. The alectinib, pharmaceutically acceptable salt thereof, or pharmaceutical composition for use according to any of claims 24 to 30, wherein the subject is aged <2 years.

32. The alectinib, pharmaceutically acceptable salt thereof, or pharmaceutical composition for use according to any of claims 24 to 30, wherein the subject is aged ≥2 to <6 years.

33. The alectinib, pharmaceutically acceptable salt thereof, or pharmaceutical composition for use according to any of claims 24 to 30, wherein the subject is aged ≥6 to <12 years.

34. The alectinib, pharmaceutically acceptable salt thereof, or pharmaceutical composition for use according to any of claims 24 to 30, wherein the subject is aged ≥12 to <18 years.

35. The alectinib, pharmaceutically acceptable salt thereof, or pharmaceutical composition for use according to any of claims 24 to 34, wherein the subject does not comprise a mutation conferring resistance to ALK inhibitors.

36. The alectinib, pharmaceutically acceptable salt thereof, or pharmaceutical composition for use according to any of claims 24 to 35, wherein the subject has not had prior exposure to an ALK inhibitor.

37. The alectinib, pharmaceutically acceptable salt thereof, or pharmaceutical composition for use according to any of claims 24 to 36, wherein the subject has had prior treatment for an ALK fusion-positive solid tumour or central nervous system tumour.

38. The alectinib, pharmaceutically acceptable salt thereof, or pharmaceutical composition for use according to any of claims 24 to 37, wherein the alectinib, or pharmaceutically acceptable salt thereof, or pharmaceutical composition is administered orally.

39. The alectinib, pharmaceutically acceptable salt thereof, or pharmaceutical composition for use according to any of claims 24 to 38, wherein the alectinib, or pharmaceutically acceptable salt thereof, or pharmaceutical composition is administered twice daily.

40. The alectinib, pharmaceutically acceptable salt thereof, or pharmaceutical composition for use according to any of claims 24 to 39, wherein the total daily dose of alectinib, or pharmaceutically acceptable salt thereof, is between 50 and 1500 mg, preferably wherein:
i. the subject is ≥40 kg and the total daily dose of alectinib, or pharmaceutically acceptable salt thereof, is between 600 and 1500 mg;
ii. the subject is ≥35 to <40 kg and the total daily dose of alectinib, or pharmaceutically acceptable salt thereof, is between 400 and 1000 mg;
iii. the subject is ≥30 to <35 kg and the total daily dose of alectinib, or pharmaceutically acceptable salt thereof, is between 300 and 800 mg;
iv. the subject is ≥25 to <30 kg and the total daily dose of alectinib, or pharmaceutically acceptable salt thereof, is between 300 and 700 mg;
v. the subject is ≥20 to <25 kg and the total daily dose of alectinib, or pharmaceutically acceptable salt thereof, is between 250 and 600 mg;
vi. the subject is ≥15 to <20 kg and the total daily dose of alectinib, or pharmaceutically acceptable salt thereof, is between 200 and 500 mg;
vii. the subject is ≥10 to <15 kg and the total daily dose of alectinib, or pharmaceutically acceptable salt thereof, is between 150 and 400 mg;
viii. the subject is ≥7.5 to <10 kg and the total daily dose of alectinib, or pharmaceutically acceptable salt thereof, is between 100 and 300 mg;
ix. the subject is ≥5 to <7.5 kg and the total daily dose of alectinib, or pharmaceutically acceptable salt thereof, is between 75 and 250 mg; or
x. the subject is ≥3.5 to <5 kg and the total daily dose of alectinib, or pharmaceutically acceptable salt thereof, is between 50 and 200 mg.

41. The pharmaceutical composition for use according to any of claims 25 to 40, wherein the pharmaceutical composition comprises alectinib hydrochloride.

42. The pharmaceutical composition for use according to any of claims 25 to 41, wherein the pharmaceutical composition is formulated as a capsule, preferably a hard capsule.

43. The pharmaceutical composition for use according to claim 42, wherein the capsule comprises alectinib hydrochloride equivalent to 20 mg or 150 mg alectinib as free base.

44. The pharmaceutical composition for use according to claim 42 or claim 43, wherein:
i. the subject is ≥40 kg and the total daily dose of alectinib, or pharmaceutically acceptable salt thereof, is between 1000 and 1400 mg, preferably the total daily dose is 1200 mg, even more preferably 600 mg BID;
ii. the subject is ≥35 to <40 kg and the total daily dose of alectinib, or pharmaceutically acceptable salt thereof, is between 700 and 900 mg, preferably the total daily dose is 760 mg, even more preferably 380 mg BID;
iii. the subject is ≥30 to <35 kg and the total daily dose of alectinib, or pharmaceutically acceptable salt thereof, is between 600 and 800 mg, preferably the total daily dose is 680 mg, even more preferably 340 mg BID;
iv. the subject is ≥25 to <30 kg and the total daily dose of alectinib, or pharmaceutically acceptable salt thereof, is between 500 and 700 mg, preferably the total daily dose is 600 mg, even more preferably 300 mg BID;
v. the subject is ≥20 to <25 kg and the total daily dose of alectinib, or pharmaceutically acceptable salt thereof, is between 500 and 600 mg, preferably the total daily dose is 540 mg, even more preferably 270 mg BID;
vi. the subject is ≥15 to <20 kg and the total daily dose of alectinib, or pharmaceutically acceptable salt thereof, is between 400 and 500 mg, preferably the total daily dose is 460 mg, even more preferably 230 mg BID;
vii. the subject is ≥10 to <15 kg and the total daily dose of alectinib, or pharmaceutically acceptable salt thereof, is between 300 and 400 mg, preferably the total daily dose is 380 mg, even more preferably 190 mg BID;
viii. the subject is ≥7.5 to <10 kg and the total daily dose of alectinib, or pharmaceutically acceptable salt thereof, is between 200 and 300 mg, preferably the total daily dose is 280 mg, even more preferably 140 mg BID;
ix. the subject is ≥5 to <7.5 kg and the total daily dose of alectinib, or pharmaceutically acceptable salt thereof, is between 150 and 250 mg, preferably the total daily dose is 200 mg, even more preferably 100 mg BID; or
x. the subject is ≥3.5 to <5 kg and the total daily dose of alectinib, or pharmaceutically acceptable salt thereof, is between 100 and 200 mg, preferably the total daily dose is 160 mg, even more preferably 80 mg BID.

45. The pharmaceutical composition for use according to any of claims 25 to 41, wherein the pharmaceutical composition is formulated as a suspension.

46. The pharmaceutical composition for use according to claim 45, wherein:
i. the subject is ≥40 kg and the total daily dose of alectinib, or pharmaceutically acceptable salt thereof, is between 600 and 800 mg, preferably the total daily dose is 718 mg, even more preferably 359 mg BID;
ii. the subject is ≥35 to <40 kg and the total daily dose of alectinib, or pharmaceutically acceptable salt thereof, is between 400 and 500 mg, preferably the total daily dose is 456 mg, even more preferably 228 mg BID;
iii. the subject is ≥30 to <35 kg and the total daily dose of alectinib, or pharmaceutically acceptable salt thereof, is between 350 and 450 mg, preferably the total daily dose is 408 mg, even more preferably 204 mg BID;
iv. the subject is ≥25 to <30 kg and the total daily dose of alectinib, or pharmaceutically acceptable salt thereof, is between 300 and 400 mg, preferably the total daily dose is 360 mg, even more preferably 180 mg BID;
v. the subject is ≥20 to <25 kg and the total daily dose of alectinib, or pharmaceutically acceptable salt thereof, is between 280 and 380 mg, preferably the total daily dose is 324 mg, even more preferably 162 mg BID;
vi. the subject is ≥15 to <20 kg and the total daily dose of alectinib, or pharmaceutically acceptable salt thereof, is between 220 and 320 mg, preferably the total daily dose is 276 mg, even more preferably 138 mg BID;
vii. the subject is ≥10 to <15 kg and the total daily dose of alectinib, or pharmaceutically acceptable salt thereof, is between 180 and 280 mg, preferably the total daily dose is 228 mg, even more preferably 114 mg BID;
viii. the subject is ≥7.5 to <10 kg and the total daily dose of alectinib, or pharmaceutically acceptable salt thereof, is between 100 and 200 mg, preferably the total daily dose is 168 mg, even more preferably 84 mg BID;
ix. the subject is ≥5 to <7.5 kg and the total daily dose of alectinib, or pharmaceutically acceptable salt thereof, is between 80 and 180 mg, preferably the total daily dose is 120 mg, even more preferably 60 mg BID;
x. the subject is ≥3.5 to <5 kg and the total daily dose of alectinib, or pharmaceutically acceptable salt thereof, is between 50 and 150 mg, preferably the total daily dose is 96 mg, even more preferably 48 mg BID.

47. Use of a therapeutically effective amount of alectinib, or a pharmaceutically acceptable salt thereof, in the manufacture of a medicament for use in treating an ALK fusion-positive solid tumour or central nervous system tumour in a subject, wherein the subject is aged <18 years.
